(19) United States
(12) Patent Application Publication
(10) Pub. No.: US 2006/0171883 A1
(43) Pub. Date: Aug. 3, 2006

(54) Detection, localization and staging of tumors using labeled activated lymphocytes directed to a tumor specific epitope

(76) Inventors: Catherine A Phillips, Grayslake (IT); Stephen B Wright, Amarillo (IT)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(21) Appl. No.: 10/530,736

(22) PCT Filed: Oct. 10, 2003
(86) PCT No.: PCT/US03/32602

§ 371 Date: Jan. 6, 2006

(30) Foreign Application Priority Data
Oct. 10, 2002 (US) 60417303

(51) Int. Cl. A61K 51/00; G01N 33/567

(52) U.S. Cl. 424/1.49; 435/7.21

(57) ABSTRACT

A Disclosed are methods for detecting and localizing a cell-specific antigen in a mammal, such as a human subject, comprising exposing peripheral blood mononuclear cells (PBMCs) of the mammal to an immunogenic peptide epitope of the antigen, under conditions for antigen-specific activation of T lymphocytes in the PBMCs, thereby producing antigen-specific T lymphocytes that at least bind to the cell-specific antigen. Labeled antigen-specific T lymphocytes are administered to the mammal, typically with-out IL-2, either intraperitoneally or intravenously. The distribution of these cells in the mammal is determined by imaging, thereby detecting and localizing cell-specific antigen in the mammal. Exposing PBMCs to the immunogenic peptide typically involves a cell-free peptide preparation and interleukin-2 (IL-2), but no additional cells such as antigen presenting cells (APC) separately pulsed with antigen. The antigen-specific T lymphocytes typically are cytolytic for cells expressing the cell-specific antigen and may comprise CD4+, CD8+, and/or CD45RO+ memory T cells.

This application claims the benefit of U.S. Provisional Patent Application No. 60/417,303, filed Oct. 10, 2002, the entirety of which is hereby incorporated herein by reference for all purposes.

### FIELD OF THE INVENTION

The present invention is in the field of clinical medicine, including diagnosis and therapy. The invention relates to the use of activated lymphocytes directed against a cell-specific antigen, such as a tumor specific epitope, their ability to migrate and attach to the tumor epitope to which they were sensitized, the ability of these cells to amplify the localization of very small tumors; and their use in identifying unknown primary tumors with a known immunogenic epitope, particularly mucin-producing adenocarcinomas.

### BACKGROUND OF THE INVENTION

Methods for imaging tissues, such as lymphatic tissues, within the intact human body are known, employing gross imaging agents or antibodies. For instance, U.S. Pat. No. 4,735,210 to Goldenberg, issued Apr. 5, 1988, discloses lymphographic and organ imaging methods and kits, particularly an improved method for lymphoscintigraphy or magnetic resonance lymphography which involves subtraction of a negative image produced using a gross imaging agent from a positive image produced with a specific antibody imaging agent. Another embodiment of the invention uses an antibody to lymphatic tissue as an imaging agent for lymphatics. A further embodiment uses a magnetic resonance image enhancing agent for magnetic resonance lymphography. Yet another embodiment uses labeled antibodies against organ antigens for scintigraphic and magnetic resonance organ imaging.

Imaging of radiolabeled cells, such as lymphocytes, in the whole human body also is well known. For example, Wagstaff, J. et al., “A method for following human lymphocyte traffic using indium-111 oxine labeling,” Clin Exp Immunol. March;43(3):435-442 (1981a), discloses a method whereby large numbers of human lymphocytes are separated from peripheral blood and labelled in vitro with indium-111 oxine. Following autologous reinjection, the distribution within the body is followed by means of serial blood samples, surface-probe counting and gamma camera imaging. The authors report that good resolution of lymphoid structures is obtained using gamma camera imaging and the changes recorded in organ distribution correlate well with data from animal models of lymphocyte migration. Thus, indium-111 oxine labelling of human lymphocytes provides a non-invasive method whereby the migratory properties of human lymphocytes can be followed. Wagstaff, J. et al, “Human lymphocyte traffic assessed by indium-111 oxine labelling: clinical observations,” Clin Exp Immunol. 43:443-449 (1981b), discloses clinical studies using indium-111 oxine labelling of human peripheral blood lymphocytes. The physiological significance of bone marrow and liver localization on gamma camera imaging is discussed and the importance of considering the surface marker characteristics of the lymphocytes under study, when interpreting results, is emphasized.

Various lymphoid cells have been used for imaging in the whole mammalian body, including different types of cells found in peripheral blood. For instance, Reynolds, C. W. et al., “Natural killer activity in the rat. IV. Distribution of large granular lymphocytes (LGL) following intravenous and intraperitoneal transfer,” Cell Immunol. 86:371-380 (1984), discloses that highly enriched populations of rat large granular lymphocytes (LGL) and T lymphocytes were prepared on discontinuous density gradients of Percoll, labeled with either ¹¹¹In-oxine or ⁵¹Cr and injected either intravenously (iv) or intraperitoneally (ip) into normal syngeneic recipients. Following iv inoculation of labeled LGL or T cells into normal recipients, a large proportion of radioactivity (18 to 33%) was recovered within minutes in the lungs. By 2 to 4 hr following transfer, significantly more LGL (13.5%) than T cells (6.4%) remained in the lungs. This difference persisted through 48 hr (5.4 vs 0.8%). Decreasing levels of radioactivity in the lungs were accompanied by corresponding increases in counts in the spleen and liver. At early time points, a significantly higher proportion of T cells was found to distribute to the spleen, while labeled LGL persisted for longer periods in the blood as well as in the lungs. Following ip inoculation into normal recipients, there was a slow clearance of radiolabeled LGL and T cells from the peritoneal cavity, with less than 20% of the radiolabel found in peripheral organs by 24 hr. These results were said to demonstrate a distribution pattern for LGL and T cells that resembles the previously reported proportions of these cells in various organs.

Radiolabeling also has been used to elucidate distribution of autologous “killer” blood monocytes infused into patients with various cancers after being made cytotoxic by in vitro incubation under various conditions, a procedure generally known as adoptive lymphocyte transfer or adoptive immunotherapy. For example, Stevenson, H. C. et al., “Fate of gamma-interferon-activated killer blood monocytes adoptively transferred into the abdominal cavity of patients with peritoneal carcinomatosis,” Cancer Res., 47:6100-6103 (1987), discloses that five patients with colorectal cancer widely metastatic to peritoneal surfaces were treated i.p. with infusions of autologous blood monocytes made cytotoxic by in vitro incubation with human gamma-interferon. The monocytes were purified by a combination of cytapheresis and counter-current centrifugal elutriation procedures; each week approximately 350 million activated monocytes were given to patients as adoptive immunotherapy by a single i.p. instillation. On the eighth cycle of treatment the trafficking of i.p. infused blood monocytes was studied in two patients by prelabeling the cells with ¹¹¹In. These activated cells became distributed widely within the peritoneal cavity. Two and 5 days after infusion their position within the peritoneum had not changed. When peritoneal specimens were obtained 36 h after ¹¹¹In-labeled monocyte infusion, labeled monocytes were demonstrated to be associated with the serosal surfaces by autoradiographic analysis. Scintiscanning structures outside the abdominal cavity revealed that ¹¹¹In-labeled monocytes infused i.p. did not traffic to other organs during the 5 days of the study. The authors concluded that i.p. adoptive transfer of autologous killer blood monocytes is an effective way of delivering these cytotoxic cells to sites of tumor burden on peritoneal surfaces in these cancer patients.

Another report on imaging of killer lymphocytes, Swift, R. I. et al., “Imaging of metastatic colorectal cancer with tumour-activated killer lymphocytes,” Lancet 337:1511-1512 (1991), discloses that lymphocytes were taken from 6 patients with metastatic colorectal cancer and cultured with cells from the patients primary tumour to produce tumour-activated killer (TAK) lymphocytes. These workers re-injected each patient with ¹¹¹In-labelled TAK cells in order to visualize metastases. Images were taken with a gamma-camera for up to 48 h after injection. Metastases were revealed as early as 4 h in the lung and as late as 48 h in the abdomen. Liver images produced ““cold”” spots corresponding to metastatic lesions. Lymph nodes were not visualised. Re-injection of TAK cells raised against autologous colorectal tumours reveals the sites of metastases. The authors also noted that TAK cells do not require IL-2 after in vivo administration.

Similarly, distribution of various types of radiolabeled autologous peripheral blood lymphocytes stimulated ex vivo by tumor cells has been determined by imaging of radiolabeled cells. Thus, Spencer R. P. and B. Mukherji, “Utilization of tumour-sensitized (‘educated’) and radiolabelled lymphocytes for tumour localization,” Nucl Med Commun. 9:783-786 (1988), discloses that, upon coculture of peripheral blood lymphocytes (PBL) in the presence of irradiated autologous tumour cells, the PBL can be sensitized to the tumour (or perhaps more correctly resensitized), as shown by in vitro cytotoxic properties. The cells can be proliferated in the presence of interleukin-2, radiolabelled with 111In and injected back into the cell donor. Using this technique, tumour deposits were localized in five out of seven patients. In an extension of this work, Mukherji, B. et al., “Imaging pattern of previously in vitro sensitized and interleukin-2 expanded autologous lymphocytes in human cancer,” Int J Rad Appl Instrum B, 15:419-427 (1988), discloses in vivo patterns of lymphocytes sensitized against autologous tumor (in vitro) that were studied in seven patients with metastatic cancer as a potential candidate for an alternative method of radioimmunodetection and adoptive immunocytotherapy. Peripheral blood lymphocytes (PBL) were either activated in Interleukin-2 (IL-2) [lymphokine activated killer (LAK) cells] or sensitized against autologous tumor cells by in vitro co-culture (IVC) and expanded in IL-2 (educated cells); both were then labeled with ¹¹¹In. Labeled autologous cells (1×10⁷-5×10⁸) were administered to patients and biodistribution studied by imaging under a gamma camera at various time intervals. In 4/7 cases, imaging with the “educated” cells showed concentrations of radioactivity at sites that correlated positively with clinically detectable metastatic tumor. By contrast, only one instance of positive uptake was seen with the LAK cells. Educated lymphocytes were cytotoxic against autologous tumor cells and the cytotoxic reactivities of the educated cells were maintained in continuous culture in IL-2 for 4-6 weeks. Evidence of accumulation of radiolabelled educated autologous cells at a significantly higher frequency than that of the LAK cells was said to suggest that in vitro expanded educated PBL might be better candidates for radioimmunodetection of human cancer, and continuous cultures of such educated autologous PBL might be sources for repeated administration of these effector cells.

Another type of lymphoid cell that has been used for adoptive immunotherapy and imaging within the human body by radiolabeling is tumor-infiltrating lymphocytes (TIL), as developed, for instance, by S. A. Rosenberg and colleagues. Thus, Fisher, B. et al., “Tumor localization of adoptively transferred indium-111 labeled tumor infiltrating lymphocytes in patients with metastatic melanoma,” J Clin Oncol. 7:250-261 (1989), discloses that lymphoid cells infiltrating into human tumors can be expanded in vitro in medium containing interleukin-2 (L2). The authors have investigated whether infused TIL labeled with indium 111(¹¹¹In) oxine can traffic and localize to metastatic deposits of tumor. Six patients with metastatic malignant melanoma who had multiple sites of subcutaneous, nodal, and/or visceral disease were the subjects of the study. The patients received cyclophosphamide 36 hours before receiving the intravenous (IV) infusion of TIL followed by IL-2 IV every eight hours. The distribution and localization of the TIL were evaluated using serial whole body gamma camera imaging, serial blood and urine samplings, and serial biopsies of tumor and normal tissue. ¹¹¹In-labeled TIL localized to lung, liver, and spleen within two hours after the infusion of activity. Activity in the lung diminished within 24 hours. As early as 24 hours after injection of ¹¹¹In-labeled TIL, localization of TIL to sites of metastatic deposits was demonstrated in all six patients using either imaging studies or biopsy specimens or both. ¹¹¹In activity in tumor tissue biopsies ranged from three to 40 times greater than activity in normal tissue. A progressive increase in the radioactive counts at sites of tumor deposit was seen. This study shows that labeled TIL can localize preferentially to tumor, and provides information concerning the possible mechanism of the therapeutic effects of TIL.

Many additional studies on adoptive immunotherapy, including comparisons of imaging with TIL and lymphoid cells of other origins have been reported. For instance, Griffith K. D., et al., “In vivo distribution of adoptively transferred indium-111-labeled tumor infiltrating lymphocytes and peripheral blood lymphocytes in patients with metastatic melanoma,” J Natl Cancer Inst. 81:1709-1717 (1989), discloses that patients with metastatic melanoma undergoing therapy with cyclophosphamide (CPM), tumor-infiltrating lymphocytes (TIL), and interleukin-2 (IL-2) were studied for the ability of their ¹¹¹In-labeled TIL or peripheral blood lymphocytes (PBL) to localize in sites of tumor using gamma camera imaging and biopsies. Nineteen infusions of radiolabeled TIL were given to 18 patients, while five patients received radiolabeled autologous PBL during TIL therapy. Clear tumor localization was seen on 13 of 18 nuclear scan series performed on ¹¹¹In-TIL recipients, while tumor was imaged in only one of four scan sequences on patients given ¹¹¹In-PBL. One patient was studied by nuclear scanning on three consecutive treatment courses of CPM, TIL, and IL-2. He initially demonstrated clear tumor localization by ¹¹¹In-TIL at several sites, then faint localization with ¹¹¹In-PBL at a single site, and subsequently positive tumor imaging on repeat ¹¹¹In-TIL infusion at multiple sites. These results confirm and expand our initial data demonstrating that human TIL transferred with CPM pretreatment and followed by IL-2 preferentially localize to tumor sites and indicate that this localization is greater for TIL than PBL.

Further imaging studies with TIL include Chin. Y., et al., “In vivo distribution of radio-labeled tumor infiltrating lymphocytes in cancer patients,” In Vivo 7:27-30 (1993), which discloses that, to evaluate the efficacy of tumor infiltrating lymphocyte in treatment, attempts were made to study the in vivo migration and distribution of these lymphocytes. Tumor-infiltrating lymphocytes isolated from five patients with malignant metastatic breast cancer or melanoma cultured and expanded in vitro with low-dose of recombinant interleukin-2 were labeled with ¹¹¹Indium-oxine and infused to the patients. A large view gamma camera was used to evaluate the distribution and localization of the infused TIL. Localization of ¹¹¹In-labeled TIL in the lungs was seen within two hours after infusion and high levels of radioactivity were observed at 24 hours in lungs, liver and spleen. The activity in the lungs diminished after 72 hours. No specific localization of ¹¹¹In-labeled TIL was observed in the metastatic sites.

Another study of TIL imaging is disclosed in Pockaj, B. A. et al., “Localization of ¹¹¹Indium-labeled tumor infiltrating lymphocytes to tumor in patients receiving adoptive immunotherapy. Augmentation with cyclophosphamide and correlation with response,” Cancer 73:1731-1737 (1994), which discloses that the adoptive transfer of interleukin-2 (IL-2)-cultured tumor infiltrating lymphocytes (TIL) can cause tumor regression in patients with metastatic melanoma. Thirty-eight patients with metastatic melanoma receiving high dose IL-2 and TIL were studied for the ability of autologous ¹¹¹In-labeled TIL to localize to metastatic tumor deposits by gamma camera imaging and biopsy. Tumor localization by ¹¹¹In-labeled TIL was seen by gamma camera imaging in 26 (68.4%) treatment courses. In a univariate analysis of factors influencing TIL traffic, cyclophosphamide administration was significantly associated with the ability to localize tumor by radionuclide imaging (P2=0.026). The authors concluded that localization in tumor may be important in the mechanism of TIL antitumor activity because no clinical responses were seen in patients who did not have their tumors imaged with ¹¹¹In-TIL, and that cyclophosphamide administration before TIL and IL-2 therapy and the administration of large numbers of TIL appear to improve the frequency of TIL localization to tumor.

Imaging of TIL is also disclosed in Dillman, R. O., “Tumor localization by tumor infiltrating lymphocytes labeled with indium-111 in patients with metastatic renal cell carcinoma, melanoma, and colorectal cancer,” Cancer Biother Radiopharm. 12:65-71 (1997). This article discloses that one issue in adoptive immunotherapy with autologous tumor infiltrating lymphocytes (TIL) is whether such cells actually migrate to sites of tumor after intravenous infusion. The authors noted that there have been several reports of tumor uptake of radiolabeled TIL in patients with metastatic melanoma, but efforts to visualize tumor with radiolabeled TIL in other tumor types reportedly have been unsuccessful. Here, eight patients with metastatic cancer (5 renal, 2 melanoma, 1 colon) received an intravenous infusion of 2 to 100 billion autologous TIL, including 50 million TIL which had been conjugated to 500 microCi Indium-111, co-administered with interleukin-2 (IL-2). One patient received 1 gm/m² of cyclophosphamide one day prior to TIL; seven patients received interferon alpha 2b for 4 days prior to receiving TIL. Total body gamma camera imaging, including single photon emission computerized tomography (SPECT), was performed at 24 and 48 hours. All eight patients had demonstrable uptake of ¹¹¹Indium-labeled TIL into one or more known sites of tumor. There were no known sites of tumor which were not imaged. Metastatic sites imaged included bone, brain, mediastinal and perihilar lymph nodes, lung and liver parenchyma, abdominal periaortic nodes, and a pelvic mass. One patient served as a negative control in that the TIL scan was negative at a time when she had no evident disease, but a few weeks later had a positive TIL scan which lead to a diagnosis of axillary recurrence. The authors concluded that uptake of radiolabeled TIL, whether CD8+ or CD4+, by metastatic renal cell carcinoma and other carcinomas was similar to that previously reported in melanoma. Pretreatment with cyclophosphamide was not a prerequisite for imaging, and TIL uptake did not predict tumor response.

Lymphocytes from peripheral blood of patients having diseases involving lymphoid tissues also have been radiolabeled for imaging in the body, without in vitro stimulation, as an aid in staging of the disease. Thus, Grimfors, G. et al., “Tumour imaging of indium-111 oxine-labelled autologous lymphocytes as a staging method in Hodgkin's disease,” Eur J Haematol. 42:276-283 (1989), discloses that following indium-111 oxine-labelled autologous lymphocyte infusion, 39 lymphocyte scintigraphies were performed in 35 patients with Hodgkin's disease (HD). Lymphocytes were obtained after leukapheresis and Lymphoprep gradient centrifugation and were further purified by an adherence step to eliminate monocytes. A median of 1.2 (0.2-3.7)×10⁹ cells were labelled with 6.7 (range 1.9-16.6) Mbq indium-111 oxine and reinfused to the patients. Gamma camera imaging was performed with a GE 400 AT. An accumulation of radioactivity was seen at 54 of 61 sites with enlarged lymph nodes. Increased radioactivity was also seen at 16 sites with no previous clinical evidence of tumour involvement. These authors suggested that the data seemed very promising and with further methodological improvement lymphoscintigraphy may prove to be an important complement in the staging procedure and follow-up of patients with HD.

Imaging of radiolabeled peripheral blood lymphocytes without in vitro activation has also been employed in other lymphatic malignancies. For instance, Muller, C., “In vivo tracing of indium-111 oxine-labeled human peripheral blood mononuclear cells in patients with lymphatic malignancies,” J Nucl Med. 30:1005-1011 (1989), discloses that in vivo migration of [¹¹¹In]oxine-labeled peripheral mononuclear cells (PMNC) was studied in 20 patients with various lymphatic malignancies and palpable enlarged lymph nodes. The maximal labeling dose of 10 microCi (0.37 MBq) [¹¹¹in]oxine/10⁸ PMNC was found not to adversely influence either cell viability or lymphocyte proliferation in vitro. For in vivo studies, 1.5×10⁹ PMNC were gained by lymphapheresis and reinjected intravenously after radioactive labeling, 150 microCi (5.55 MBq). The labeling of enlarged palpable lymph nodes was achieved in three out of three patients with Hodgkin's disease and in five out of five with high-malignant lymphoma, whereas three out of seven patients with low malignant lymphoma and no patient with chronic lymphatic leukemia had positive lymph node imaging. The authors thus concluded that PMNC retain their ability to migrate after [¹¹¹In]oxine labeling and that these cells traffic to involved lymph nodes of some, but not all hematologic malignancies.

Distribution of human PBLs in animals and humans after infusion by different routes has also been studied. For instance, Nelson, H. et al., “Regional and systemic distribution of anti-tumor x anti-CD3 heteroaggregate antibodies and cultured human peripheral blood lymphocytes in a human colon cancer xenograft,” J Immunol. 145:3507-3515 (1990), dicscloses that anti-tumor antibody (317G5) covalently coupled to an anti-CD3 antibody (OKT3) produces a heteroaggregate (HA) antibody that can target PBL to lyse tumor cells expressing the appropriate tumor Ag. The i.v. and i.p. distribution of radiolabeled HA antibody 317G5×OKT3 and of radiolabeled cultured human PBL were studied in athymic nude mice bearing solid intraperitoneal tumor established from the human colon tumor line, LS174T. Mice were injected with ¹²⁵I-labeled HA antibody, ¹²⁵I-labeled anti-tumor mAb, or ¹¹¹In-labeled PBL, and at designated timepoints tissues were harvested and measured for radioactivity. ¹²⁵I-317G5×OKT3 localized specifically to tumor sites. The major difference in radioactivity levels observed between i.v. and i.p. administration of ¹²⁵I-317G5×OKT3 was an increase in hepatic radioactivity after i.v. HA antibody administration. Autoradiography confirmed that anti-tumor x anti-CD3 HA antibodies localized specifically to intraperitoneal tumor; that i.p. administered HA antibodies penetrated tumor directly; and that i.v. administered HA antibodies distributed along tumor vasculature. Cultured human PBL distributed in moderate concentrations to intraperitoneal tumor when administered i.p., but not when administered i.v. The authors speculated that poor localization of i.v. injected PBL to tumor may reflect species disparity in homing receptors and/or endothelial ligands, a problem which may be overcome with a syngeneic model. They concluded that these results suggest that regional therapy with HA antibodies and PBL may offer advantages over systemic therapy for initial clinical trials.

Lymphocytes from tissues other than tumor masses (i.e., TIL) or peripheral blood also have been studied for adoptive immunotherapy, including distribution by imaging of radiolabeled lymphocytes or antibodies thereto. For instance, U.S. Pat. No. 5,814,295, to Martin, Jr. et al., issued Sep. 29, 1998, discloses determination of lymph nodes enriched in tumor reactive cells, their proliferation and their use in adoptive cellular therapy. In particular, the invention is directed to a method for reliably determining lymph nodes enriched in tumor reactive cells, e.g., CD4+tumor-specific lymphocytes. This method includes the steps of administering to a patient an effective amount of a radiolabeled locator which specifically binds a marker produced by or associated with neoplastic tissue. Time is permitted to elapse following the administration for the radiolabeled locator to preferentially concentrate in any neoplastic tissue and for unbound radiolabeled locator to be cleared, so as to increase the ratio of photon emissions from neoplastic tissue to background photon emissions in the patient. After the time has elapsed, the patient is accessed with a radiation detection probe for determining lymph node sites exhibiting accretion of the radiolabeled locator by detecting with the probe elevated levels of radiation at the lymph node sites. The lymph node sites exhibiting such elevated levels of radiation are removed and subjected to gross visual analysis, though such sites alternatively may be subjected to histological analysis. Those determined and removed lymph nodes that also are determined to be tumor-free by gross observation or free of gross metastatic disease are selected and cultured to proliferate tumor reactive cells. The selected lymph nodes are subjected to mitogenic stimulation. The lymph nodes are cultured in the presence of Interleukin-2, anti-CD3 monoclonal antibody, and neoplastic tissue which may be autologous or allogeneic tumor. This patent also discloses that two patients (Nos. 3 and 4) given such stimulated lymph node cells have shown evidence of tumor regression with and without exogenous IL-2, that extensive immunologic data has been collected, including cell trafficking studies using ¹¹¹In-labelled cells (patient 5), but that it is too early to draw any definitive conclusions.

Recent adoptive immunotherapy methods have employed T cells from various sources that are stimulated in vitro by antigen-presenting cells, particularly dendritic cells that have been pulsed with various sources of antigen. For instance, Peng, L. et al., “Helper-independent, L-selectin^{low} CD8+ T cells with broad anti-tumor efficacy are naturally sensitized during tumor progression,” J Immunol. 165:5738-5749 (2000), discloses that the authors recently reported on a CD4(+) T cell subset with low L-selectin expression (CD62L^{low}) in tumor-draining lymph nodes (TDLN) which can be culture activated and adoptively transferred to eradicate established pulmonary and intracranial tumors in syngeneic mice, even without coadministration of IL-2. This report extends these studies to characterize the small subset of L-selectin^{low} CD8(+) T cells naturally present in TDLN of mice bearing weakly immunogenic tumors. Isolated L-selectin^{low} CD8(+) T cells displayed the functional phenotype of helper-independent T cells, and when adoptively transferred could consistently eradicate, like L-selectin^{low} CD4(+) T cells, both established pulmonary and intracranial tumors without coadministration of exogenous IL-2. Culture-activated L-selectin^{low} CD8(+) T cells did not lyse relevant tumor targets in vitro, but secreted IFN-gamma and GM-CSF when specifically stimulated with relevant tumor preparations. The authors concluded that these data indicate that even without specific vaccine maneuvers, progressive tumor growth leads to independent sensitization of both CD4(+) and CD8(+) anti-tumor T cells in TDLN, phenotypically L-selectin^{low} at the time of harvest, each of which requires only culture activation to unmask highly potent stand-alone effector function.

Adoptive immunotherapy of human patients with T cells primed by autologous dendritic cells pulsed with tumor antigens from various sources also has been reported. Thus, Santin, A. D. et al., “Development and therapeutic effect of adoptively transferred T cells primed by tumor lysate-pulsed autologous dendritic cells in a patient with metastatic endometrial cancer,” Gynecol Obstet Invest. 49:194-203 (2000), describes a 65-year-old woman with a large surgically unresectable and chemoresistant liver metastasis of endometrial carcinoma who was treated by infusion with peripheral blood T cells stimulated with tumor lysate-pulsed autologous dendritic cells (DC). Extensive in vitro characterization of the DC-activated T cells included phenotypic analysis, cytotoxicity, and intracellular cytokine secretion. High cytotoxicity was observed against autologous tumor cells, but not against NK-sensitive K562 cells, autologous Con-A lymphoblasts, or autologous Epstein-Barr virus-transformed lymphoblastoid cells. Blocking studies demonstrated that lytic activity was HLA class I restricted. Two-color flow cytometric analysis revealed that a significant proportion of CD8+ T cells was also CD56+, and analysis of intracellular IFN-gamma and IL-4 expression suggested a type 1 cytokine bias. The patient was treated by three infusions of tumor-specific T cells at 3- to 4-week intervals, and in vivo distribution of the T cells was followed by ¹¹¹In oxine labeling and serial gamma camera imaging. Tumor localization and accumulation of labeled lymphocytes was consistently detected at serial time points following each injection. However, deep infiltration of the large tumor mass by activated T cells was minimal, as evaluated in 3 dimensions by single photon emission computerized tomography (SPECT) imaging. Clinically, stabilization of the large liver metastasis was obtained during treatment. Collectively, these results were said to indicate that tumor-specific CD8+ cytotoxic T-cell responses can be generated in patients with endometrial cancer, and to suggest that T-cell immunotherapy may be of therapeutic value in patients harboring metastatic disease. As can be seen in FIG. 5, localization of radioactivity [of ¹¹¹In oxine labeled CD8+ cytotoxic T-cells] around the tumor predominated over that of the remaining normal liver parenchyma, when compared with ^{99m}Tc-sulfur colloid scintigraphy.

Human mucin 1 (MUC1, also called MUC-1) is an epithelial mucin glycoprotein that is overexpressed in 90% of all adenocarcinomas including breast, lung, pancreas, prostate, stomach, colon, and ovary. MUC1 is a target for immune intervention, because, in patients with solid adenocarcinomas, low-level cellular and humoral immune responses to MUC1 have been observed, which are not sufficiently strong to eradicate the growing tumor. For instance, Hiltbold, E. M., et al., “Naturally processed class II epitope from the tumor antigen MUC1 primes human CD4+ T cells,” Cancer Res. 58:5066-5070 (1998), reports that epithelial cell mucin MUC1 is expressed on adenocarcinomas in an underglycosylated form that serves as a tumor antigen in breast, pancreatic, ovarian, and other tumors, and that two predominant MUC1-specific immune responses are found in patients: CD8+ CTLs, which recognize tandemly repeated epitopes on the MUC1 protein core, and IgM antibodies. Asserting that there have been no prior reports of MUC1-specific CD4+ T-helper cells in cancer patients, and show that MUC1-specific CD4+ T cells are neither deleted nor tolerized and that CD4+ T cell responses can be generated when an appropriate soluble form of MUC1 is used. Naive CD4+ T cells from healthy donors were primed in vitro to a synthetic MUC1 peptide of 100 amino acids, representing five unglycosylated tandem repeats, presented by dendritic cells. They produced IFN-gamma and had moderate cytolytic activity. The authors also identified one core peptide sequence, PGSTAPPAHGVT, that elicits this response when it is presented by HLA-DR3.

MUC1 is unusual in that MHC-unrestricted TCR recognition of a tumor-specific peptide epitope on this antigen can occur. Thus, Magarian-Blander, J. et al., “Intercellular and intracellular events following the MHC-unrestricted TCR recognition of a tumor-specific peptide epitope on the epithelial antigen MUC1,” J. Immunol. 160:3111-3120 (1998), discusses the functional and molecular parameters involved in direct TCR recognition of a tumor-specific peptide epitope on MUC1. This peptide epitope is tandemly repeated and recognized on the native molecule rather than processed and bound to the MHC. As taught in Magarian-Blander, J. et al., T lymphocytes typically recognize antigenic peptides that are presented to them within the groove of self MHC molecules on the surface of APC. This MHC-restricted recognition is mediated through the TCR/CD3 complex. Recognition of the antigenic peptide by the TCR/CD3 complex results in the activation of the T cell via a cascade of signal-transduction events. Activation of the T cell ultimately results in proliferation and transcriptional activation of a variety of genes that lead to the release of cytokines, expression of new surface molecules, and maturation of effector function.

Magarian-Blander, J. et al. also note that they have reported previously on MHC-unrestricted β T cells that recognize a peptide epitope on MUC1, a type I transmembrane glycoprotein that is expressed on the surface of ductal epithelial cells as well as carcinomatous cells of the same origin. The bulk of its extracellular domain is composed of a tandemly repeated 20-amino acid sequence that contains the T cell epitope. The MHC-unrestricted recognition of the MUC1 tandem repeat epitope is blocked by Abs to the TCR and CD3 complex, showing that this recognition is TCR mediated. These workers have proposed that MUC1 tandem repeats present a dense array of unprocessed epitopes directly to the TCR as rigid structures that bypass the need for presentation by MHC molecules. Structural studies of the MUC1 tandem repeat protein core using synthetic peptide analogues have confirmed that the T cell epitope assumes a stable ordered structure that forms a loop protruding past the extended β-turn helix structure of the polypeptide core.

More recently, activation of MUC1 antigen-specific anti-tumor lymphocytes in vitro by incubation with an epitope-presenting peptide alone, without presentation of peptide using separately prepared antigen-presenting cells, has been reported for certain MUC1 epitopes. Thus, Wright S. E. et al., “Cytotoxic T lymphocytes from humans with adenocarcinomas stimulated by native MUC1 mucin and a mucin peptide mutated at a glycosylation site,” J Immunother. 23:2-10 (2000), disclose that MUC1 mucin peptides stimulated cytotoxic T lymphocytes (CTL) from humans with adenocarcinomas. Peripheral blood mononuclear cells (PBMCs), tumor-draining lymph node (TDLN) cells, or tumor-infiltrating lymphocytes (TIL) were stimulated using mono-nuclear cells from humans with adenocarcinomas of breast or ovary, respectively, using (a) a native MUC1 mucin tandem repeat peptide of 20 amino acids (MUC1-mtr1) plus recombinant human interleukin-2 (IL-2), (b) the mutated (T3N) MUC1-mtr1 plus IL-2, or (c) immobilized anti-CD3 plus IL-2, or (d) IL-2 alone. The CTL stimulated by each of these four conditions were predominately CD4+. However, the CTL stimulated by either the native MUC1-mtr1 or (T3N) MUC1-mtr1 showed 5-10 times greater cytotoxicity of a breast cancer cell line that expresses MUC1 compared to CTL stimulated by either anti-CD3+ IL-2 or IL-2 alone. Each incubation condition generated CTL with different variable beta gene families of T-cell receptors, implying an oligoclonal expansion of a limited CTL repertoire for each. Thus, peptide-stimulated T cells showed expression of cytotoxic cells, which was not induced by nonspecific (anti-CD3 or IL-2) stimulation. Further, these authors reported that cytotoxicity of the mucin-peptide stimulated cell lines was non-HLA restricted, presumably at the level of HLA class I and II for the PBLC from a donor with breast cancer and at least class I for TIL from a donor with ovarian cancer. This work was extended by the present inventors (Wright, S. E. . . . , Phillips, C. A., et al., “Adoptive immunotherapy of mucin1 expressing adenocarcinomas with mucin1 stimulated human peripheral blood mononuclear cells,” Int J Mol Med. 9:401-404 (2002)) to show that mucin1 stimulated hematopoietic mononuclear cells (M1SHMC) from patients with breast cancer, adoptively transferred to non-obese diabetic, severe combined immunodeficient (NOD SCID) mice, extended survival in a therapy model of gross adenocarcinoma and prevented tumor growth in a model of minimal disease. M1SHMC exhibited specific lysis of a human breast adenocarcinoma cell line expressing mucin1, MCF-7 and produced interferon gamma. M1SHMC were injected intraperitoneally (IP) in NOD SCID mice after gross, palpable tumors appeared after MCF-7 were injected subcutaneously (SC). Survival was increased as compared to no M1SHMC controls. However tumors eventually regrew in all mice. To determine whether minimal disease (MD) could be controlled, NOD SCID were injected with MCF-7 cells, and on the same day, injected IP with M1SHMC. The M1SHMC injected mice were protected from tumor growth. These results imply that M1SHMC can prolong survival, but not cure NOD SCID mice bearing gross palpable adenocarcinomas. However in a minimal disease model tumor growth was prevented.

U.S. Pat. No. 6,600,012 to Agrawal et al., issued Jul. 29, 2003, relating to lipid-modified muc-1 derivatives, discloses a method for generating a mixture of activated T-cells by combining a plurality of peripheral blood lymphocytes (PBLs) with an antigen-loaded liposome to produce antigen-loaded PBLs, and combining the antigen-loaded PBLs with a naive, anergic or memory T-cell, to produce an activated T-cell. Such activation is carried out in vivo or in vitro. The antigen-loaded PBLs and activated T-cells, prepared according to the methods of invention, are said to have use as cellular vaccines for treatment of cancer and viral diseases. This patent and references cited therein teach that dendritic cells (DCs) were initially considered to be potential APCs to prime naive T-cells, and that DCs have been used as APCs for in vitro stimulation of primary antigen-specific CTL responses. It has been suggested that DCs are capable of intensive aggregation with unprimed T-cells and express a high density of accessory molecules, such as B7.1 and B7.2. which are critical for stimulation of naive resting T-cells. However, Agrawal et al. further teach that DCs are not good candidates for (1) determining the immunogenicity of various peptides for immunotherapy and (2) stimulation of T-cells for expansion for adoptive cell therapy. In this regard, the prior art relates to generation of antigen-specific CD8.sup.+CTL responses using DCs. According to Agrawal et al., the disclosed invention provides a method for generating activated T-cells, comprising: (a) combining liposome-encapsulated peptide antigen with a plurality of peripheral blood lymphocytes to produce antigen-loaded antigen-presenting cells; (b) combining naive or anergic T-cells with said antigen-loaded antigen-presenting cells; (c) isolating activated T-cells from the combination of step (b). In a further embodiment, the invention provides such a method wherein said activated T-cells are T helper cells and provides a method wherein said activated T-cells are cytotoxic T-cells. A preferred embodiment relates to the production of a population of MUC-1 peptide specific, activated CD4+ and CD8+ T-cells, which are generated in vitro by activating naive T-cells with PBLs (as APCs) that were previously loaded with liposome encapsulated peptide antigens. In another embodiment, the invention relates to the generation of activated T-cells using naive T-cells, memory T-cells, and anergic T-cells, or a mixture of all three cell types, along with liposome-encapsulated antigen and autologous whole peripheral blood lymphocytes (PBLs) as antigen presenting cells.

Use of lymphocytes for imaging of non-tumor cell specific antigens also has been disclosed. For instance, Mazzoni, G. et al, “Indium-labeled presensitized T cells for diagnosis of graft rejection,” J Surg Res. 52:85-88 (1992) discloses that the aim of the reported experiment was to test a safe, noninvasive method for necessary, accurate diagnosis of early allograft rejection. Heart-lung allograft was performed heterotopically using Brown Norway (BN) rats as the donor and Lewis (LEW) rats as the recipient. T cell suspensions were prepared from lymphnodes of specifically sensitized LEW rats that had acutely rejected full-thickness BN skin graft. Cell count was adjusted 50×10(6) cells/ml. The suspension was incubated in vitro with ¹¹¹In oxide (1 mCi-mi). An aliquot of labeled cell suspension containing 40×10⁶ cells and a total radioactivity of 200 mCi was administered intravenously to each animal 3 and 6 days after heart-lung transplant. The traffic of T cells was followed in vivo and in isolated organs under large field view gamma camera. The gamma camera revealed radioactivity on the graft starting Postoperative Day 5 when the heart was actively beating; no radioactivity was revealed at the site of the isografted organs. The histology showed mild to moderate cellular infiltration parallel to the grade of radioimaging intensity. The injection of indium-labeled presensitized T cells was able to detect the rejection process in an early phase when there are no clinical symptoms of rejection and/or the rejection cascade can be reversed.

Various other radiolabel-based methods are known for imaging of cells within the mammalian body. For instance, Anders, G. T. et al., “SPECT gallium-67 scanning in early human immunodeficiency virus (HIV) infection. Failure of scanning abnormalities to correlate with immunologic staging,” Clin Nucl Med. 15:295-302 (1990), discloses the use of gallium scanning in the treatment of patients with AIDS. Also, Korf, J., et al., “Divalent cobalt as a label to study lymphocyte distribution using PET and SPECT,” J Nucl Med. 39:836-841 (1998), teaches that PET and SPECT allow the study of the distribution of lymphocytes in living humans, provided that these cells are adequately prelabeled ex vivo, and further describes the potential of divalent cobalt isotopes (⁵⁵Co²⁺, half-life 17.5 hr for PET; ⁵⁷Co²⁺, half-life 270 days for SPECT) for labeling lymphocytes. Still further, U.S. Pat. No. 5,840,859 to Lambert et al., issued Nov. 24, 1998, discloses (aminostyryl)pyridinium compounds for radiolabeling cell membranes with, for instance, a radioisotope of iodine, i.e., ¹²³I, ¹²⁵I, or ¹³¹I; or a chelating group comprising one equivalent of a metallic radioisotope such as ¹¹¹In or ^{99m}Tc, chelated by a polycarboxylic acid. Lambert et al. teach that autologous lymphocytes labeled in accord with the disclosed method could be used for in vivo lymphocyte tracking and clinical imaging of lymphatic malignancies, and suggest that the disclosed compounds may replace ¹¹¹Indium-oxine as the preferred agent with which to label cultured lymphocytes for imaging metastatic melanoma prior to adoptive immunotherapy.

U.S. Pat. No. 6,146,614 to Rubin et al., issued Nov. 14, 2000, is directed to a method for determining lymphocyte distribution and trafficking in mammals using imaging. Either a labeled ligand capable of interacting specifically with the lymphocytes of the mammal is administered to the mammal so that the labeled ligand interacts in vivo with the lymphocytes, resulting in labeled lymphocytes, or, the labeled ligand is contacted with the lymphocytes in vitro so that the labeled ligand interacts with the lymphocytes resulting in labeled lymphocytes, and these labeled lymphocytes are administered to the mammal. The distribution or trafficking of the labeled lymphocytes in the mammal is determined by imaging. Methods for diagnosing the degree of progression of a disease in a mammal by determining the mammal's lymphocyte distribution or trafficking pattern, for monitoring the response to a therapy in mammal having a disease, for evaluating the ability of an agent to alter the distribution or trafficking of lymphocytes, and for identifying an agent useful for treating a mammal having a disease, are also described. In certain embodiments, the mammal has a disease, e.g., an HIV infection, an autoimmune disease, an infectious disease, or a malignancy. The lymphocytes can be, e.g., B cells or T cells. For certain embodiments, it is preferred that the lymphocytes are CD4-positive cells or CD8-positive cells. The ligand can be, e.g., an antibody, e.g., a polyclonal antibody or a monoclonal antibody, e.g., anti-CD4 monoclonal antibody, an antibody fragment, a recombinant antibody, a peptide, a peptide mimetic, a carbohydrate or a glycoprotein.

BRIEF SUMMARY OF THE INVENTION

The present invention features a method for detecting and localizing a cell-specific antigen in a mammal by administration of labeled antigen-specific lymphocytes, preferably activated T cells obtained from peripheral blood mononuclear cells of the mammal by exposing such cells to a peptide that displays an immunogenic epitope of the cell-specific antigen and determining the distribution of the labeled lymphocytes in the mammal by imaging. This invention is based in part on the following operative elements appreciated by the inventors:

radiolabeled activated lymphocyte populations directed against a tumor specific epitope are useful for diagnostic approaches of this invention;

such cells have an ability to migrate and attach to the tumor epitope to which they were sensitized;

such cells have an ability to amplify the localization of very small tumors (for instance, ≦2 mm in diameter); and

such cells are useful for identifying unknown primary tumors with a known immunogenic eptiope, such as mucin-producing adenocarcinomas which can detected using anti-mucin peptide cytotoxic T lymphocytes (CTL), that is, T cells activated with an immunogenic peptide derived from the amino acid sequence of a mucin polypeptide or another tumor antigen.

Accordingly, one embodiment of the invention process comprises:

Lymphoid populations derived from patients' blood or other lymphoid containing compartments would be stimulated against a specific tumor epitope, such as mucin;

The activated lymphoid cells would be assessed for their ability to kill or recognize specific tumors in vitro;

The activated populations of lymphoid cells would be radiolabeled or “tagged” with Indium oxine or other similar radioisotope in vitro;

The radiolabeled cells would be delivered either intravenously or intraperitoneally (abdominal cavity tumors);

The biodistribution would be monitored by nuclear medicine for 48 hours and images analyzed for presence of “hot spots” as in ProstaScint® prostate screen technology;

SPECT nuclear medicine images would be fused with CT or MRI images to determine if there was demonstrable tumor at these sites, and these images may be compared with PET images for additional confirmation. The inventors further appreciated that use of these non-invasive diagnostic modalities would prevent unnecessary time lost to surgical recuperation, e.g., after exploratory surgery that identified no metastases, and facilitate the use of more effective modalities if metastatic disease is detected, e.g., in new locations after treatment has been initiated. Diagnostic methods using positron emission tomography (PET) alone have been shown to dramatically alter clinical treatment. The inventors therefore believe that this present methodology alone, or combined with other technologies such as PET, would allow earlier detection as well as better staging of cancers.

Initial clinical studies on the invention method have been disclosed in Phillips, C. A., et al., “Trafficking as a consideration in adoptive immunotherapy: How does the route of administration affect the delivery of cytotoxic T lymphocytes to tumor?” J. Immunol. 25(6):S34 (November/December 2002), as follows:
Adoptive immunotherapy using specific cytotoxic T lymphocytes (CTL) is an experimental therapy intended to destroy residual tumor after conventional surgery, radiation, and chemotherapy. The migration patterns of the adoptively transferred CTL preparations, administered by different routes, have been minimally investigated in patients. We have monitored the movement in vivo of ¹¹¹Indium-labeled CTL preparations stimulated against tumor mucin peptide. The biodistribution of these cells is very different when administered intravenously versus intraperitoneally. Preparations of activated lymphocytes were administered in a small series of breast cancer patients (two with and two without tumor) and five ovarian cancer patients with recurrent tumor (BB IND 8620). The protocol was written for two infusions per month (one radiolabeled and one not labeled, over a 4-month period). The biodistribution of the intravenously administered CTL preparation in the breast cancer patients was typical of an indium oxine leukocyte scan. Quantification of the biodistribution over time revealed that non-cytotoxic T and non-specific cytotoxic cells did not traffic to tumor. The details of cell migration over time will be shown. In the ovarian cancer patients, the pattern was quite remarkable. The intraperitoneally infused, radiolabeled CTL preparation established a recognizable pattern at the first image, which refined itself over the next several days and is unique to each patient. The movement out of the peritoneum is rapid, approximately 10% at 1 hour and approximately 30% at 98 hours. Radiolabeled CTL preparations localized to known tumors (from CT scans) and to areas not previously identified as tumor metastases, intra- and extra-peritoneal. The biodistribution of radiolabeled CTL preparations of specific cases for breast and ovarian cancer patients and the fusion of CT scans and SPECT images for an ovarian case will be shown. Recommendations for CTL delivery will be discussed in detail.

In view of the above, it is an object of the invention to provide a safe, effective and easy, preferably non-invasive, method for detecting cell-specific antigen by detecting activated antigen-specific lymphocyte distribution and trafficking in an individual.

It is another object of the invention to utilize imaging techniques to determine localization of a cell-specific antigen from an individual's activated lymphocyte distribution and trafficking patterns.

It is yet another object of the invention to evaluate the progression of disease in an individual by determining localization of a cell-specific antigen from an individual's activated lymphocyte distribution and trafficking patterns.

Still another object of the invention is to evaluate the efficacy of therapies for a disease by assessing effects of the therapies on localization of a cell-specific antigen from activated lymphocyte distribution and trafficking patterns in individuals.

Accordingly, in one aspect the present invention provides a method for detecting and localizing a cell-specific antigen in a mammal, such as a human being, comprising the steps of: (a) obtaining peripheral blood mononuclear cells from the mammal; (b) exposing the peripheral blood mononuclear cells to an immunogenic peptide that displays an immunogenic epitope of the cell-specific antigen, under conditions such that T lymphocytes in the peripheral blood mononuclear cells undergo antigen-specific activation (thereby producing activated antigen-specific T lymphocytes that bind to the cell-specific antigen); (c) labeling the antigen-specific T lymphocytes with a label that is detectable by imaging; (d) administering the labeled antigen-specific T lymphocytes to the mammal, and (e) determining the distribution of the labeled antigen-specific T lymphocytes in the mammal by imaging, thereby detecting and localizing the cell-specific antigen in the mammal.

In some embodiments of the invention method, above, step (b) of exposing the peripheral blood mononuclear cells to the immunogenic peptide is performed in the presence of interleukin-2 (IL-2) to facilitate activation of T cells.

Advantageously, step (b) of exposing the peripheral blood mononuclear cells (PBMC) to the immunogenic peptide is performed by adding a cell-free preparation of the peptide to the peripheral blood mononuclear cells without adding additional cells to the PBMC prior to step (d) in which the labeled antigen-specific T lymphocytes are administered to the mammal. Thus, in contrast to previously disclosed methods of T cell activation, for instance, in U.S. Pat. No. 6,600,012 to Agrawal et al., wherein a population of MUC-1 peptide specific, activated CD4+ and CD8+ T-cells was generated in vitro by activating naive T-cells in PBMC with peripheral blood leukocytes (PBLs) acting as antigen presenting cells (APCs). These PBLs were previously loaded with liposome encapsulated peptide antigens in a separate culture and then added to cultures of naive T-cells from PBMC, thereby increasing the complexity of the T cell activation process and also the risk of loss due to contamination or other causes inherent in manipulating multiple cell cultures.

Also advantageous in the present invention is the use of antigen-specific T lymphocytes that are cytolytic for cells that express the cell-specific antigen. Thus, cytolytic (or, more generally, cytotoxic) T cells (CTLs) may bind more effectively than noncytolytic antigen specific T cells to cells bearing a targeted cell-specific antigen; however, in contrast to adoptive immunotherapy applications, the present diagnostic methods do not absolutely require T cells capable of effective killing of antigen-bearing target cells. Thus, the activated antigen specific T cells of the invention method may comprise CD4+ lymphocytes or CD8+ lymphocytes or mixtures thereof. Activated antigen specific cells of the invention method also may comprise memory T cells, particularly CD45RO+ memory T cells. Another advantage of the invention method for producing antigen-specific T lymphocytes, via exposure of T cells from PBMCs to cell-free antigen, such as a polypeptide or peptide displaying an epitope of the target antigen, is that such antigen-specific T lymphocyte may comprise negligible amounts of natural killer (NK) cells ( e.g., less than about 10%, preferably less than about 6% and more preferably less than about 3%, as shown, for instance, by the percentage of cells having a CD3−, CD8−, CD56+ phenotype).

In some embodiments, advantageously the step (d) of administering the labeled antigen-specific T lymphocytes to the mammal is performed without administering cytokines, particularly IL-2, to the mammal with the T lymphocytes or thereafter, before performing step (e) of determining the distribution of the labeled antigen-specific T lymphocytes in the mammal. Thus, whereas adoptive immunotherapy with T cells, such as TIL cells, typically requires high doses of cytokines, particularly IL-2, to be administered to the patient with and after the activated cytotoxic T cells, to maintain their cytotoxicity, when used for diagnostic purposes in the present invention the activated T cells need not initially be, or be maintained in a cytotoxic state of activation. By using T cells activated according to the invention method, therefore, the additional cost and potential side effects of high dose IL-2 administration that is required for adoptive transfer of other types of activated T cells may be avoided.

In some embodiments of the present invention method, step (d) of administering the labeled antigen specific T lymphocytes to the mammal comprises administering the lymphocytes intraperitoneally. Thus, as disclosed in Phillips, C. A., et al., supra, the migration patterns of the adoptively transferred CTL preparations stimulated against tumor mucin peptide are very different when administered intravenously versus intraperitoneally. The biodistribution of the intravenously administered CTL preparation in the breast cancer patients was typical of an indium oxine leukocyte scan. For instance, Reynolds, C. W. et al., supra, teaches that, following iv inoculation of labeled LGL or T cells into normal recipients, a large proportion of radioactivity (18 to 33%) was recovered within minutes in the lungs. Decreasing levels of radioactivity in the lungs were accompanied by corresponding increases in counts in the spleen and liver. Similarly, Chin. Y., et al., supra, reported that localization of ¹¹¹In-labeled TIL in the lungs was seen within two hours after infusion and high levels of radioactivity were observed at 24 hours in lungs, liver and spleen. The activity in the lungs diminished after 72 hours, but no specific localization of ¹¹¹In-labeled TIL was observed in metastatic sites. Finally, Swift, R. I. et al., supra, reported that ¹¹¹In-labelled tumor activated killer (TAK) cells revealed metastases as early as 4 h in the lung and as late as 48 h in the abdomen, but liver images produced “cold” spots corresponding to metastatic lesions, presumably due to the high background of cells in normal liver tissue.

In contrast to the general findings with intravenous administration of various lymphocytes, the present inventors have found that intraperitoneally infused, radiolabeled CTL preparations established a recognizable pattern at the first image, which refined itself over the next several days and is unique to each patient. The movement out of the peritoneum is rapid, approximately 10% at 1 hour and approximately 30% at 98 hours. Radiolabeled CTL preparations localized to known tumors (from CT scans) and to areas not previously identified as tumor metastases, intra- and extra-peritoneal.

In yet other embodiments of the invention method, step (d) of administering the labeled antigen specific T lymphocytes to said mammal comprises administering the lymphocytes intravenously. Advantageously, in such embodiments, administering the lymphocytes intravenously further comprises administering a glycoconjugate to the mammal method such that trafficking of the lymphocytes is altered compared to administering the lymphocytes without administering the glycoconjugate. Thus, International Application No. PCT/US93/07834, published as WO 03/077864 A2 on Mar. 14, 2003 (hereby incorporated herein in its entirety, by reference), discloses methods for directing cells to target cells. Some embodiments of these methods comprise the steps of administering, either simultaneously or sequentially, a carbohydrate presenting molecule (e.g., glycoconjugate) and a cell to the mammal. In these methods, glycoconjugates, especially asialoglycoconjugates, including asialo plasma proteins such as asialoorosomucoid (asialo alpha-(1)-acid glycoprotein), are thought to transiently bind the hepatic asialoglycoprotein receptor and thereby competitively inhibit attachment of cells, including lymphocytes and particularly CD4+ cells, which bear asialodeterminants bound by those hepatic receptors. Without wishing to be bound by theory, the inventors believe that hyposialylated and desialylated proteins/glycoconjugates (also called asialoglycoconjugates) and cells which bear similar determinants are bound or “trapped” in the liver as a consequence of binding to the hepatic asialoglycoprotein receptors. Occupation of the receptor by the asialoglycoconjugate inhibits sequestration of the cells bearing similar determinants of interest in the liver. Accordingly, in the present invention method, administration of an asialoglycoconjugate, such as asialoorosomucoid prior to or concurrently with intravenous administration of antigen specific T lymphocytes, particularly CD4+ lymphocytes, prevents such lymphocytes from accumulating in normal lung and liver tissues, thereby reducing the background of labeled cells in such tissue and enhancing detection of antigen-bearing cells, such as tumor cells, in these tissues. In addition, the above PCT disclosure shows that glycoconjugates of the disclosed invention prevent infused cells from concentrating in the alveolar vasculature. Accordingly, administration of asialoglycoconjugate, such as orosomucoid, prior to or concurrently with intravenous administration of antigen specific T lymphocytes, particularly CD4+ lymphocytes, also prevents such lymphocytes from accumulating in normal lung tissues, thereby reducing the background of labeled cells this tissue, while enhancing delivery of cells to the liver and spleen.

In another aspect, the present invention provides a method for detecting and localizing a cell-specific antigen in which the cell-specific antigen is a tumor-specific antigen. Advantageously, the antigen is a tumor-specific mucin, such as human mucin1 (MUC-1), and the peptide immunogen displays an epitope of MUC-1. MUC-1 (also called MUC1) is an epithelial mucin glycoprotein that is overexpressed in 90% of all adenocarcinomas including breast, lung, pancreas, prostate, stomach, colon, and ovary. Advantageously, the invention method using an immunogenic peptide epitope of MUC-1 provides T cells comprising CD4+ lymphocytes that exhibit MHC unrestricted cytotoxicity for cells bearing the MUC-1 epitope. Thus, Magarian-Blander, J. et al., supra, discuss the MHC-unrestricted TCR recognition of a tumor-specific peptide epitope on MUC-1 Further, Wright S. E. et al., supra, discloses that MUC1 mucin peptides stimulated cytotoxic T lymphocytes (CTL) in peripheral blood mononuclear cells (PBMCs) from humans with adenocarcinomas. Thus, peptide-stimulated T cells showed expression of cytotoxic cells, which was not induced by nonspecific (anti-CD3 or IL-2) stimulation. Further, these authors reported that cytotoxicity of the mucin-peptide stimulated cell lines was non-HLA restricted (i.e., MHC unrestricted). The advantage of using an immunogen that provides antigen specific T lymphocytes that exhibit MHC unrestricted cytotoxicity is that a single such immunogen can be used in the invention method to produce antigen specific T lymphocytes from any patient, regardless of MHC background.

Other instances of Ag-specific MHC-unrestricted recognition have also been described, as disclosed in Magarian-Blander, J. et al. and references cited therein. For instance, MHC-unrestricted T cells specific for mycobacterial Ags have been isolated from the synovial fluid of patients with rheumatoid arthritis, and from mice immunized with Mycobacterium tuberculosis. MHC-unrestricted T cells have also been isolated that are specific for Ags such as Ig Ids on B cell tumors, a herpesvirus glycoprotein, and nonpeptide prenyl pyrophosphates. Ag-specific, MHC-unrestricted, β T cells have also been described for complex proteins such as avidin and myelin basic protein, as well as for a nonpeptide Ag such as the heme moiety of hemoglobin. Several studies have also described arsonate- and fluorescein-specific T cells that can recognize Ag in the absence of MHC molecules. Carbohydrate-specific MHC-unrestricted T cells also have been generated that were specific for the carbohydrate moiety on glycosylated peptides derived from the vesicular stomatitis virus nucleoprotein.

Advantageously, for localization of cells expressing MUC-1 according to the present invention, the immunogenic peptide displays an epitope of MUC-1 comprising an amino acid sequence that is a circular permutation of a MUC-1 sequence that comprises the sequence (expressed in conventional single letter code): (SEQ ID NO: 1) PDTRP. Advantageously, the peptide has the amino acid sequence (SEQ ID NO: 2) GSTAPPAHGVTSAPDTRPAP. Other MUC-1 immununogenic peptides and derivatives thereof that can be used in the present invention are disclosed elsewhere, for instance, in U.S. Pat. No. 6,600,012 to Agrawal et al., including but not limited to, a MUC-1 peptide that generates an antigen-specific T cell response and comprises a sequence of from about 7 to about 20 amino acids of the amino acid sequence STAPPAHGVTSAPDTRAPGSTAPP (SEQ ID NO. 3).

Also advantageously, the immunogenic peptide used in the invention method is a liposome-encapsulated peptide or a MUC-1 peptide that is covalently modified with a lipid moiety, for instance, when the invention method uses activated T cells generated as taught by Agrawal et al., with a method comprising: (a) combining liposome-encapsulated peptide antigen with a plurality of peripheral blood lymphocytes to produce antigen-loaded antigen-presenting cells; (b) combining naive or anergic T cells with said antigen-loaded antigen-presenting cells; and (c) isolating activated T-cells from the combination of step (b).

The label used for detection of lymphocytes according to the present invention may be any label known in the art for detecting cells within the mammalian body. Advantageously, the label is selected from the group consisting of a gamma emitter, a positron emitter, a magnetic material, a density based contrast material, and mixtures thereof. When the label is a gamma emitter it may be selected from the group consisting of indium-111, technetium-99m, technetium-99, iodine-123, and mixtures thereof. Advantageously, the label is indium-111, typically in the form of indium oxine.

The imaging technology for localization of labeled lymphocytes in the invention method may be selected from the group consisting of radioimaging, magnetic resonance imaging, positron emission tomographic and X-ray computed tomographic imaging, depending, of course, on the nature of the label. Further, the imaging may be performed in a single scan or in serial scans. Advantageously, imaging used in the invention method comprises a total body scan of the mammal.

In some embodiments imaging comprises at least two separate scans, wherein each separate scan is selected from the group consisting of radioimaging, magnetic resonance imaging (MRI), positron emission tomographic (PET) and X-ray computed tomographic (CT) imaging. Advantageously, in such embodiments imaging data obtained from two or more separate scans are compared, for instance, by a process in which data from multiple scans are fused into a single display image. For example, U.S. Pat. No. 4,735,210 to Goldenberg, issued Apr. 5, 1988, discloses lymphographic and organ imaging methods and kits, particularly an improved method for lymphoscintigraphy or magnetic resonance lymphography which involves subtraction of a negative image produced using a gross imaging agent from a positive image produced with a specific antibody imaging agent. More recently, U.S. Pat. No. 6,490,476, to Townsend et al., issued Dec. 3, 2002, teaches a combined PET and X-ray CT tomograph and method for using same for acquiring CT and PET images sequentially in a single device, overcoming alignment problems due to internal organ movement, variations in scanner bed profile, and positioning of the patient for the scan. The tomograph acquires functional and anatomical images which are accurately co-registered, without the use of external markers or internal landmarks.

### DETAILED DESCRIPTION OF THE INVENTION

One purpose of this invention is to detect smaller tumors earlier than is now possible by standard imaging techniques as well as to find tumors in which the original primary tumor cannot be found easily. Another benefit is that recurrences could be better staged so that the optimum care plan can be devised for the patient avoiding unnecessary surgery. This technique could be used to verify findings such as MRI, CT, PET, and laboratory tests.

In one aspect, therefore, the present invention provides a method for detecting and localizing a cell-specific antigen in a mammal, such as a human being, comprising the steps of: (a) obtaining peripheral blood mononuclear cells from the mammal; (b) exposing the peripheral blood mononuclear cells to an immunogenic peptide that displays an immunogenic epitope of said cell-specific antigen, under conditions such that T lymphocytes in the peripheral blood mononuclear cells undergo antigen-specific activation (thereby producing antigen-specific T lymphocytes that bind to the cell-specific antigen); (c) labeling the antigen-specific T lymphocytes with a label that is detectable by imaging; (d) administering the labeled antigen-specific T lymphocytes to the mammal, and (e) determining the distribution of the labeled antigen-specific T lymphocytes in the mammal by imaging, thereby detecting and localizing the cell-specific antigen in the mammal. This method is particularly useful in human subjects, especially patients having a disease or condition involving abnormal expression of a cellular antigen, such as a tumor specific antigen.

1. Activated Antigen-Specific T Lymphoctes

Obtaining peripheral blood mononuclear cells (PBMC) from the mammal for use in the present invention may generally be done by any conventional method known in the art. For instance, the “buffy coat” is collected from peripheral blood samples using a method such as Ficoll-Hypaque gradient centrifugation to separate peripheral blood lymphocytes from other components. See, for instance, the techniques described at pages 7.0.5 through 7.1.5 of CURRENT PROTOCOLS IN IMMUNOLOGY (John E. Coligan, ed., John Wiley & Sons, New York, 1991), hereby incorporated by reference.

The invention method further comprises activating antigen specific T cells in PBMCs from the mammal, particularly by exposing the PBMCs to an immunogenic peptide that displays an immunogenic epitope of the cell-specific antigen, under conditions such that T lymphocytes in the PBMCs undergo antigen-specific activation (thereby producing activated antigen-specific T lymphocytes that bind to the cell-specific antigen). An “activated” T lymphocyte or T cell, as used herein, is undergoing mitosis and/or cell division. An activated T lymphocyte may be a T helper (T_{H}) cell or a cytotoxic T cell (cytotoxic T lymphocyte (CTL or T_{C})). Activation of a naive T-cell may be initiated by exposure of such a cell to an antigen presenting cells (APC) (which contains antigen/MHC complexes) and to a molecule such as IL-1, IL-2, IL-12, IL-13, γ-IFN, and similar lymphokines. The antigen/MHC complex interacts with a receptor on the surface of the T cell (T cell receptor (TCR)). Golub et al., eds. Immunology: a Synthesis, Chapter 2: “The T-cell Receptor” (1991). Accordingly, in some embodiments of the present invention method, step (b) of exposing the peripheral blood mononuclear cells to the immunogenic peptide is performed in the presence of interleukin-2 (IL-2) to facilitate activation of T cells.

Advantageously, step (b) of exposing the peripheral blood mononuclear cells (PBMC) to the immunogenic peptide is performed by adding a cell-free preparation of the peptide to the peripheral blood mononuclear cells without adding additional cells to the PBMC prior to step (d) in which the labeled antigen-specific T lymphocytes are administered to the mammal. Thus, in contrast to previously disclosed methods of T cell activation, for instance, in U.S. Pat. No. 6,600,012 to Agrawal et al., wherein activated CD4+ and CD8+ T-cells were generated in vitro by activating naive T-cells with peripheral blood leukocytes (PBLs) acting as antigen presenting cells (APCs), the present invention advantageously avoids the need for antigen presenting cells by use of T lymphocyte precursors in PBMC of subjects expressing a tumor specific antigen. Thus, without being bound by theory, the inventors believe that PBMC of subjects expressing a tumor specific antigen, particularly a MUC-1 tumor antigen, contain precursors of antigen specific T lymphocytes that can be activated to at least bind to the tumor antigen by direct exposure to a immunogenic peptide epitope of that antigen, without requiring an APC to present the peptide. For instance, T lymphocytes in PBMC of subjects expressing a tumor specific antigen may have experienced “priming” in the host that expresses tumor specific antigen. In this specification, “priming” is used to mean exposing an animal (including a human) or cultured cells to antigen, in a manner that results in activation and/or memory. The generation of CD4+ and CD8+ T cell responses against a target antigen is usually dependent upon in vivo priming, either through natural infection or through deliberate immunization. In any event, a detailed clinical protocol used for producing activated antigen specific T lymphocytes in clinicial trials of the invention method is presented in Example 1, below.

Alternatively, activated antigen specific T lymphocytes used in the invention method can be produced by activation of naive T cells, never exposed to the cell specific antigen, by in vitro exposure to APC loaded with an antigen, particularly with a peptide epitope of the targeted cell specific antigen, or an immunogenic derivative of such a peptide epitope. For instance, the APCs may be PBLs that were previously loaded with liposome encapsulated peptide antigens in a separate culture and then added to cultures of naive T-cells from PBMC, as described in U.S. Pat. No. 6,600,012 to Agrawal et al.

Also advantageous in the present invention is the use of antigen-specific T lymphocytes that are cytolytic for cells that express the cell-specific antigen. Thus, cytolytic (or, more generally, cytotoxic) T cells (CTLs) may bind more effectively than noncytolytic antigen specific T cells to cells bearing a targeted cell-specific antigen; however, in contrast to adoptive immunotherapy applications, the present diagnostic methods do not absolutely require T cells capable of effective killing of antigen-bearing target cells. Thus, the activated antigen specific T cells of the invention method may comprise CD4+ lymphocytes or CD8+ lymphocytes or mixtures thereof. Activated antigen specific cells of the invention method also may comprise memory T cells, particularly CD45RO+memory T cells. As used in this specification “memory T cells,” also known as “memory phenotype” T cells, is used to designate a class of T lymphocytes that have previously encountered a peptide antigen but are now resting and are capable of being activated. Memory T cells are T cells which have been exposed to antigen and then survive for extended periods in the body without the presence of stimulating antigen. However, these memory T cells respond to “recall” antigens. In general, memory T cells are more responsive to a “recall” antigen, when compared with the naive T cell response to peptide antigen. Memory cells can be recognized by the presence of certain cell-surface antigens, such as CD45R0, CD58, CD11α, CD29, CD44 and CD26, which are markers for differentiated T cells. Memory T cells are isolated by techniques well-known to the skilled artisan. For instance, briefly, the total T cell population is isolated, followed by fluorescence activated cell sorting (FACS) using anti-CD45R0, anti-CD44 or anti-CD26 monoclonal antibodies. See Hollsberg et al., Cellular Immunology 149:170 (1993); Bruno et al., Immunity 2:37 (1995); and J Immunol. 150 (part 1):3119 (1993).

Another advantage of the invention method for producing antigen-specific T lymphocytes according to the present invention, via exposure of T cells from PBMCs to cell-free antigen, such as a polypeptide or peptide displaying an epitope of the target antigen, is that such antigen-specific T lymphocyte may comprise negligible amounts of natural killer (NK) cells (e.g., less than about 10%, preferably less than about 6% and more preferably less than about 3%, as shown, for instance, by the percentage of cells having a CD3-, CD8-, CD56+ phenotype). For instance, Wright S. E. et al., J. immunother. 23:2-10 (2000) reported 3-6% of the total cells in activated preparations exhibited the above NK phenotype. Further, antigen-specific T cells produced according to the invention method can be stored frozen after activation, for periods of at least several months, thereby providing a uniform source of T cells for reproducible labeling and imaging conditions for multiple localizations of antigen in the same patient, for instance, to follow the progress of treatment over a period of weeks or months.

2. Administration of Activated T Lymphocytes

In some embodiments, advantageously the step (d) of administering the labeled antigen-specific T lymphocytes to the mammal is performed without administering cytokines, particularly IL-2, to the mammal with the T lymphocytes or thereafter, before performing step (e) of determining the distribution of the labeled antigen-specific T lymphocytes in the mammal. Alternatively, as in adoptive immunotherapy with T cells such as TIL, LAK or NK (but not TAK) cells, that require cytokine support in vivo, administration of cytokines, particularly IL-2, may be included concurrently with and/or after the antigen specific lymphocytes are administered. For example, Pockaj, B. A. et al., supra, discloses adoptive transfer of interleukin-2 (IL-2)-cultured tumor infiltrating lymphocytes (TIL) in patients with metastatic melanoma receiving high dose IL-2 and TIL.

Administration of the labeled lymphocytes into the mammal can be accomplished by a variety of methods, including, e.g., injection, infusion, deposition, implantation, oral ingestion or topical administration. Preferably, administration is by injection. Injections can be, e.g., intravenous, intradermal, subcutaneous, intramuscular or intraperitoneal. In some embodiments of the present invention method, step (d) of administering the labeled antigen specific T lymphocytes to the mammal advantageously comprises administering the lymphocytes intraperitoneally. Thus, as disclosed in Phillips, C. A., et al., supra, the migration patterns of the adoptively transferred CTL preparations stimulated against tumor mucin peptide are very different when administered intravenously versus intraperitoneally.

The biodistribution of the intravenously administered CTL preparation in breast cancer patients, according to the present invention, was typical of an indium oxine leukocyte scan. For instance, Reynolds, C. W. et al., supra, teaches that, following intravenous inoculation of labeled LGL or T cells into normal recipients, a large proportion of radioactivity (18 to 33%) was recovered within minutes in the lungs. Decreasing levels of radioactivity in the lungs were accompanied by corresponding increases in counts in the spleen and liver. Similarly, Chin. Y., et al., supra, reported that localization of ¹¹¹In-labeled TIL in the lungs was seen within two hours after infusion and high levels of radioactivity were observed at 24 hours in lungs, liver and spleen. The activity in the lungs diminished after 72 hours, but no specific localization of ¹¹¹In-labeled TIL was observed in metastatic sites. Finally, Swift, R. I. et al., supra, reported that ¹¹¹In-labelled tumor activated killer (TAK) cells revealed metastases as early as 4 h in the lung and as late as 48 h in the abdomen, but liver images produced “cold” spots corresponding to metastatic lesions, presumably due to the high background of cells in normal liver tissue.

In contrast to the general findings with intravenous administration of various lymphocytes, the present inventors have found that intraperitoneally infused, radiolabeled CTL preparations established a recognizable pattern at the first image, which refined itself over the next several days and is unique to each patient. The movement out of the peritoneum is rapid, approximately 10% at 1 hour and approximately 30% at 98 hours. Radiolabeled CTL preparations localized to known tumors (from CT scans) and to areas not previously identified as tumor metastases, intra- and extra-peritoneal.

Intraperitoneal administration of activated lymphocytes according to the invention is therefore particularly useful for, but not limited to, subjects such as cancer patients, particularly ovarian cancer patients, having intraperitoneal tissue masses expressing the target cell antigen, such as metastases expressing a tumor-specific antigen. For instance, the invention method could be used prior to intraperitoneal surgery, to identify even metastases that may be too small to otherwise detect non-invasively, during follow up evaluation after surgery before beginning chemotherapy, and during or after a course of chemotherapy, to evaluate effectiveness of the intervention and indicate the need for additional or different therapy modalities based on lack of response of even small remaining tumor masses.

In yet other embodiments of the invention method, step (d) of administering the labeled antigen specific T lymphocytes to said mammal comprises administering the lymphocytes intravenously. Advantageously, in such embodiments, administering the lymphocytes intravenously further comprises administering a glycoconjugate to the mammal method such that trafficking of the lymphocytes is altered compared to administering the lymphocytes without administering the glycoconjugate. Thus, International Application No. PCT/US93/07834, published as WO 03/077864 A2 on Mar. 14, 2003 (hereby incorporated herein in its entirety, by reference), discloses methods for directing cells to target cells.

Glycoconjugates suitable for use in the present invention may be generally represented by the general formula P-(S)x-Gal wherein P is a peptide residue of a human serum glycoprotein and S is a sugar residue of a human serum glycoprotein; x is an integer from 1 to 100 and Gal is galactose residue. The glycoconjugates may be partially or completely asialylated. Especially useful glycoconjugates include fetuins, asialofetuins, orosomucoids and asialoorosomucoids.

The glycoconjugates may be administered to the mammal in any time frame relative to administering the antigen specific cells. They may be administered before, after or simultaneously with the administration of the cells. In a typical embodiment, the glycoconjugates are administered prior to the cell. The glycoconjugates may be administered via any suitable route. In preferred embodiments, they are administered parenterally, and more preferably, intravenously to the mammal. Accordingly, in the present invention method, administration of an asialoglycoconjugate, such as asialoorosomucoid prior to or concurrently with intravenous administration of antigen specific T lymphocytes, particularly CD4+ lymphocytes, prevents such lymphocytes from accumulating in normal lung and liver tissues. Additionally, administration of a sialoglycoconjugate, such as orosomucoid, prior to or concurrently with intravenous administration of antigen specific T lymphocytes according to the invention, particularly CD4+ lymphocytes, also prevents such lymphocytes from accumulating in normal lung tissues while enhancing delivery of cells to the liver and spleen.

3. Immunogenic Epitopes and Antigens

In another aspect, the present invention provides a method for detecting and localizing a cell-specific antigen in which the cell-specific antigen is a tumor-specific antigen. Advantageously, the antigen is a tumor-specific mucin, such as human mucin1 (MUC-1), and the peptide immunogen displays an epitope of MUC-1. MUC-1 (also called MUC1) is an epithelial mucin glycoprotein that is overexpressed in 90% of all adenocarcinomas including breast, lung, pancreas, prostate, stomach, colon, and ovary. Advantageously, the invention method using an immunogenic peptide that displays an epitope of MUC-1 provides T lymphocytes comprising CD4+ lymphocytes that exhibit MHC unrestricted cytotoxicity for cells bearing the MUC-1 epitope. Thus, Magarian-Blander, J. et al., supra, discuss the MHC-unrestricted TCR recognition of a tumor-specific peptide epitope on MUC-1 Further, Wright S. E. et al., supra, discloses that MUC1 mucin peptides stimulated cytotoxic T lymphocytes (CTL) in peripheral blood mononuclear cells (PBMCs) from humans with adenocarcinomas. Thus, peptide-stimulated T cells showed expression of cytotoxic cells, which was not induced by nonspecific (anti-CD3 or IL-2) stimulation. Further, these authors reported that cytotoxicity of the mucin-peptide stimulated cell lines was non-HLA restricted (i.e., MHC unrestricted). The advantage of using an immunogen that provides antigen specific T lymphocytes that exhibit MHC unrestricted cytotoxicity is that a single such immunogen can be used in the invention method to produce antigen specific T lymphocytes from any patient, regardless of MHC background.

Advantageously, for localization of cells expressing MUC-1 according to the present invention, the immunogenic peptide displays an epitope of MUC-1 comprising an amino acid sequence that is a circular permutation of a MUC-1 sequence that comprises the sequence (expressed in conventional single letter code): (SEQ ID NO: 1) PDTRP. Advantageously, the immunogenic peptide has the amino acid sequence (SEQ D NO: 1) GSTAPPAHGVTSAPDTRPAP. Other MUC-1 immunogenic peptides and derivatives thereof that can be used in the present invention are disclosed elsewhere, for instance, in U.S. Pat. No. 6,600,012 to Agrawal et al., and U.S. Pat. No. 6,344,203 to Sandrin et al., issued Feb. 5, 2002, disclosing peptide mimicks of MUC1 or other cancer peptides which can be included in cancer vaccines and used in the present methods for cancer patients.

Other instances of Ag-specific MHC-unrestricted recognition have also been described, as disclosed in Magarian-Blander, J. et al. and references cited therein. For instance, MHC-unrestricted T cells specific for mycobacterial Ags have been isolated from the synovial fluid of patients with rheumatoid arthritis, and from mice immunized with Mycobacterium tuberculosis. MHC-unrestricted T cells have also been isolated that are specific for Ags such as Ig Ids on B cell tumors, a herpesvirus glycoprotein, and nonpeptide prenyl pyrophosphates. Ag-specific, MHC-unrestricted, β T cells have also been described for complex proteins such as avidin and myelin basic protein, as well as for a nonpeptide Ag such as the heme moiety of hemoglobin. Several studies have also described arsonate- and fluorescein-specific T cells that can recognize Ag in the absence of MHC molecules. Carbohydrate-specific MHC-unrestricted T cells also have been generated that were specific for the carbohydrate moiety on glycosylated peptides derived from the vesicular stomatitis virus nucleoprotein.

a. Generally Useful Antigens

Antigen specific MHC class II and class I restricted CD4+ and CD8+ T-cell responses are important host immune responses against a variety of pathogenic conditions. Of particular interest, therefore, is the generation of an antigen specific T-cell response. As used in this specification, an “antigen specific” T-cell response is a T cell response (proliferative, cytotoxic, cytokine secretion) to a given antigenic stimulus, such as a peptide, which is not evident with other stimuli, such as peptides with different amino acid sequences (control peptides). The responsiveness of the T cell is measured by assessing the appearance of cell surface molecules that are characteristic of T-cell activation, including, but not limited to CD25 and CD69. Such assays are known in the art.

The present methods apply generally to a great variety of antigens. These antigens may be of nearly any chemical constitution, as long as they are able to elicit a T cell-specific immune response; they may contain at least one T cell-specific epitope. Exemplary antigens can be derived from peptides, carbohydrates, lipids and especially combinations thereof. Particularly important antigens are peptides, lipopeptides and glycopeptides. Idiotypic and antiidiotypic antigens are specifically included.

Antigens against which it would be highly advantageous to use the subject methods include tumor antigens. Tumor antigens are usually native or foreign antigens which are correlated with the presence of a tumor. Inasmuch as tumor antigens are useful in differentiating abnormal from normal tissue, they are useful not only in diagnosis, but also as a target for therapeutic intervention. Thus, the use of the present methods to generate a T cell-specific immune response against tumor antigens is an important aspect of the invention.

Tumor antigens are well known in the art. Indeed, several examples are well-characterized and are currently the focus of great interest in the generation of tumor-specific therapies. Non-limiting examples of tumor antigens are carcinoembryonic antigen (CEA), prostate specific antigen (PSA), melanoma antigens (MAGE, BAGE, GAGE), in addition to the aforementioned mucins, such as MUC-1.

MUC-1 mucin antigen has been recognized as a potential immunotherapy target to generate immunity against a number of adenocarcinomas. Thus, one embodiment of the invention relates to use in the invention method of a “MUC-1 derivative” which is capable of binding to either or both class I and class II molecules on the surface of an APC. “MUC-1 derivatives” are typically peptides or peptide-based, including, but not limited to, MUC-1 peptides recited above. A MUC-1 derivative may be a fragment of the MUC-1 protein. Such fragments may be glycosylated or unglycosylated. In accordance with the present invention, fragments within the invention can be obtained from purified MUC-1 or MUC-1 produced by recombinant DNA methodology, using methods that include digestion with proteases, such as pepsin or papain. Of course, MUC-1 fragments also may be made directly by recombinant methods. In addition, MUC-1 fragments encompassed by the present invention can be synthesized using an automated peptide synthesizer such as those supplied commercially by Applied Biosystems, Multiple Peptide Systems and others, or they may be produced manually, using techniques well known in the art. See Geysen et al., J. Immunol. Methods 102: 259 (1978). Additional MUC-1 derivatives suitable for use in the invention methods are described in Agrawal et al., supra. Moreover, one or more amino acids of the core sequence may be altered, preferably in a conservative manner known in the art, such that the requisite T cell-activating activity is maintained. Typical substitutions may be made among the following groups of amino acids: (a) G, A, V, L and I; (b) G and P; (c) S, C, T, M; (d) F, Y, and W; (e) H, K and R; and (f) D, E, N, and Q. Some preferred substitutions may be made among the following groups: (i) S and T; (ii) P and G; a (iii) A, V, L and I.

As described above, these preferred MUC-1 derivatives may be glycosylated or partially glycosylated according to methods known in the art. Moreover, it is contemplated that MUC-1 and MUC-1 derivatives can be modified with large molecular weight polymers, such as polyethylene glycols. In addition, lipid modifications are preferred because they may facilitate the encapsulation or interaction of the derivative with liposomes. Exemplary lipid moieties useful for this purpose include, but are not limited to, palmitoyl, myristoyl, stearoyl and decanoyl groups or, more generally, any C₂ to C₃₀ saturated, monounsaturated or polyunsaturated fatty acyl group. Also illustrative of a MUC-1 derivative suitable for use with the present invention is a non-peptide “mimetic,” i.e., a compound that mimics one or more functional characteristics of the MUC-1 protein. Mimetics are generally water-soluble, resistant to proteolysis, and non-immunogenic. Conformationally restricted, cyclic organic peptides which mimic MUC-1 can be produced in accordance with known methods described, for example, by Saragovi, et al., Science 253: 792 (1991). “MUC-1 carbohydrate derivatives” are also contemplated. Such a derivative, as used herein, refers to a glycopeptide which retains the immunostimulatory characteristic of MUC-1 derivatives. Such a carbohydrate derivative may include all or part of the carbohydrate that is attached to the MUC-1 protein. Mimetics that mimic at least one property of MUC-1 carbohydrate may also be used.

The skilled artisan will recognize that other antigens may be used for the generation of activated T-cells. Examples of such antigens include, but are not limited to, non-self (foreign) peptide antigens, and peptide antigens from a virus, tumor, bacterium or other parasite.

b. Identification of Other Useful Antigens

While whole antigens useful in the present methods may be identified using recognized methodologies for measuring various T cell responses, it is of interest to generate a more specific response, associated with a particular epitope. This approach allows the use of much smaller, and thus more economically produced, antigenic stimuli. Hence, preferred antigens are small molecules, typically peptides or peptide derivatives on the order of less than about 100 amino acids and usually less than about 60 amino acids.

According to methods known in the art, once a native (large) antigen has been identified, its antigenicity can be further refined to one or a few specific epitopes. One classic method involves proteolytic treatment of the large antigen to derive smaller antigens. In addition, fragments of protein antigens can be produced by recombinant DNA techniques and assayed to identify particular epitopes. Moreover, small peptides can be produced by in vitro synthetic methods and assayed. As an alternative to the random approach of making parts of the intact antigen then assaying them, a more biologically relevant approach is possible. Specifically, since antigenic fragments which bind to MHC class I and/or class II molecules are of particular importance, one exemplary approach is to isolate the MHC molecules themselves and then to isolate the peptides associated with them. Generally, this method works well for further defining particularly useful epitopes of tumor antigens.

In a typical method, either primary tumor cells or a cell line expressing the antigen of interest are provided. In addition, it will be recognized that phagocytic antigen presenting cells (or any APC), such as macrophages, may be fed large antigens (or portions thereof) and thus act as the starting material for these methods. The MHC class I or class II molecules can be isolated from these starting cells using known methods, such as antibody affinity (MHC-specific antibodies) and chromatographic techniques. Isolated MHC molecules are then treated to release bound peptides. This may be accomplished by treatment with agents that disrupt the interactions between the bound peptide and the MHC molecule, for example, detergent, urea, guanidinium chloride, divalent cations, various salts and extremes in pH). The peptides released can be further purified using conventional chromatographic and antibody affinity (using antigen-specific antibody) methodologies. The purified peptides may then be subjected to sequence and structural determinations, using for example peptide sequencing, gas chromatography and/or mass spectroscopy. In this manner the sequences/structures of the most prevalent peptide epitopes associated with class I and/or class II molecules may be determined. Supplied with this sequence/structural information, permutations of the determined sequence can be made, as detailed above, and assayed using known T-cell assays.

4. Cell Labeling and Imaging

The label used for detection of lymphocytes according to the present invention may be any label known in the art for detecting cells within the mammalian body. Conveniently, indium-111, typically in the form of indium oxine, is used to directly label lymphocytes using conventional methods well known in the art and described in references cited herein. However, various other radiolabel-based methods known for imaging of cells within the mammalian body also can be used, such as gallium-67 detection via SPECT, as disclosed by Anders, G. T. et al., supra, divalent cobalt detection using PET or SPECT as disclosed by Korf, J., et al., supra, or (aminostyryl)pyridinium compounds for radiolabelling cell membranes, for instance, a radioisotope of iodine, i.e., ¹²³I, ¹²⁵I, or ¹³¹I; or a chelating group comprising one equivalent of a metallic radioisotope such as ¹¹¹In or ^{99m}Tc, chelated by a polycarboxylic acid, as taught by Lambert et al., supra.

Alternatively, activated T cells used in the invention method may be labeled indirectly, using a labeled ligand that binds specifically to the activated T cells, as taught by Rubin et al., supra, wherein either a labeled ligand capable of interacting specifically with the lymphocytes administered to the mammal so that the labeled ligand interacts in vivo with the lymphocytes, resulting in labeled lymphocytes, or, the labeled ligand is contacted with the lymphocytes in vitro and the resulting labeled lymphocytes are administered to the mammal. The label in the labeled ligand can be, e.g., a gamma emitter, e.g., indium-111, technetium-99m, technetium-99 or iodine-123, a positron emitter, e.g., fluorine-18, carbon-11, or iodine-124, a magnetic material, e.g., gadolinium, superparamagnetic substances, or hydrated iron oxide particles, or a density based contrast material.

The distribution of the labeled lymphocytes in the body is determined by an imaging technique. By imaging is meant the detection of the distribution of the label in the body by non-invasive means. The imaging technology used in the invention method may be selected from the group consisting of radioimaging, magnetic resonance imaging, positron emission tomographic and X-ray computed tomographic imaging, depending, of course, on the nature of the label. Further, the imaging can consist of single or serial scans, and can be total or partial body scans of the mammal. Advantageously, imaging used in the invention method comprises a total body scan of the mammal, particularly SPECT imaging of ¹¹¹In-labeled lymphocytes.

Preferably, the dose of the labeled lymphocytes administered to the mammal is about equivalent to the amount of lymphocytes present in about 50 to about 75 cubic centimeters of the subject's whole blood such that the appropriate amount of radioactivity is administered. The amount of radioactivty depends upon the isotope used and can by determined by one skilled in the art without undue experimentation. For example, it is generally preferred to use about 1.5 to about 3.0 mCi/dose of indium-111, about 10 to about 30 mCi/dose of technetium-99, about 5 to about 10 mCi/dose of iodine-123, about 5 to about 10 mCi/dose iodine-124, about 10 to about 20 mCi/dose fluorine-18 and about 20 to about 30 mCi/dose carbon-11. In certain embodiments, multiple administration of the labeled lymphocytes can be used.

In some embodiments imaging comprises at least two separate scans, wherein each separate scan is selected from the group consisting of radioimaging, magnetic resonance imaging (MRI), positron emission tomographic (PET) and X-ray computed tomographic (CT) imaging. Advantageously, in such embodiments imaging data obtained from two or more separate scans are compared, for instance, by a process in which data from multiple scans are fused into a single display image. For example, U.S. Pat. No. 4,735,210 to Goldenberg, issued Apr. 5, 1988, discloses lymphographic and organ imaging methods and kits, particularly an improved method for lymphoscintigraphy or magnetic resonance lymphography which involves subtraction of a negative image produced using a gross imaging agent from a positive image produced with a specific antibody imaging agent.

More recently, U.S. Pat. No. 6,490,476, to Townsend et al., issued Dec. 3, 2002, teaches a combined PET and X-ray CT tomograph and method for using same for acquiring CT and PET images sequentially in a single device, overcoming alignment problems due to internal organ movement, variations in scanner bed profile, and positioning of the patient for the scan. The tomograph acquires functional and anatomical images which are accurately co-registered, without the use of external markers or internal landmarks.

The timing after administration of the labeled lymphocytes for a scan can be minutes, hours, days, weeks or months. The particular timing depends upon many factors, including, e.g., the type of label, the amount of label, the behavior of the lymphocytes, and the disease condition. The timing can be determined by one of ordinary skill in the art employing such factors and using no more than routine experimentation.

5. Applications of the Invention Method

Aside from particular applications described above, the invention method can be used for diagnosing the degree of progression of a disease in a mammal where the progression of the disease can be determined by changes in the distribution of a cell- or pathogen-specific antigen. For instance, the disease can be a viral infection, e.g., an HIV infection, or other infectious disease, an autoimmune disease, e.g., rheumatoid arthritis, multiple sclerosis, inflammatory bowel disease, systemic lupus erythematosis, or psoriasis, or a malignancy, e.g., myeloma, lymphoma, leukemia or a solid tumor, including tumors described above. The method can be used in a mammal having lymphocytes that can be activated by an immunogenic peptide epitope of the involved cell- or pathogen-specific antigen expressed in such a disease is provided. The distribution or trafficking patterns of the labeled lymphocytes are compared to a standard, generated either in the same mammal at a different time, or in another mammal not suffering from the disease, so as to diagnose the degree of progression of the disease. The invention is particularly useful for diseases in which immunomodulating therapy might be useful, including adoptive immunotherapy using activated T lymphocytes of the invention method.

Another aspect of the invention is a method for monitoring the response to a therapy in a mammal having a disease. The response of the mammal to the treating step is monitored, by determining whether the therapy alters the distribution or trafficking pattern of the activated antigen-specific lymphocytes by imaging.

Another aspect of the invention is a method for identifying an agent useful for treating a mammal having a disease, e.g., an HIV infection, an autoimmune disease, an infectious disease, or a malignancy, by determining whether the agent alters the distribution or trafficking pattern of the activated antigen-specific lymphocytes by imaging.

### EXAMPLE 1

### Clinical Procedures for Activating and Harvesting Activated T Lymphocytes

### CTL Generation Standard Operating Procedure

On Day 0: Patient's aphoresis bag and name labels are received from Coffee Blood Center as well as a sample contained in an extra piece of sealed tubing. This is released into a snap-cap vial to be taken to HCC for a CBC (complete blood count). Volume of aphoresis bag is noted. A patient label is attached to the culture bag and ‘labeled with trial #-patient#-aphoresis 4. A separate patient notebook is started with the first aphoresis. All pertinent data, information and results done in this lab are recorded in this book.

Sufficient AIM-V media is warned in a 37° C. water bath, 2-3 liters. Otily fresh1 unopened bottles are used. Once opened that bottle is designated for that patient only. To facilitate removing the aphoresis product from the aphoresis bag, the volume of the aphoresis bag is subtracted from 1000 ml and that volume of AIM-V will be transferred to the culture bag.

AIM-V media is transferred to the culture bag to by attaching a luer-lock 60 ml syringe to the appropriate port on the culture bag. All other ports are closed. The aphoresis bag is then emptied into the culture bag using transfer tubing, piercing the aphoresis bag and the culture bag. Using a SEBRA heat sealer, the tubing closest to the culture bag is sealed four times. The tubing is cut within the third seal from the culture bag.

Using the WBC count from the CRC as the number of cells/mi and multiplying by the volume of the aphoresis bag, the (total number of cells) in the aphoresis bag is determined. Dividing this number by 1000 ml gives the (cell concentration in the culture bag). The maximum volume of the culture bag is 2000 ml. Since the cells are to be started at 2×10ˆcells/ml, the maximum number of cells needed is 4000×10 (4×10ˆ09). If the total number of cells in the bag does not exceed 4×10⁹, then no cells are removed, If the total number of cells exceeds 4×10ˆ9 then the appropriate volume is removed.(Total # of cells In bag—4×10⁹ cells to be removed. Divide (cells to be removed) by the (cell concentration in the culture bag) to determine volume to be drawn off.

If cells do not need to be removed initially:

AM media to the culture bag with a 60 ml Luer Lock syringe, its plunger removed and replaced slightly, attached to the appropriate bag port. If more than I liter is to be added, an entire bottle of AIM-V may be poured into the syringe (now acting as a funnel) with care taken to avoid spillage. If less than a liter is to be added, a sterile, individually wrapped 50 ml pipet can be used to transfer the media from bottle to the syringe/funnel. The culture bag is rocked gently, side to side and front to back, periodically to mix the contents. If cells do need to be removed initially:

A Luer Lock 60 ml syringe is attached to the appropriate port, with its plunger in place, The culture bag is rocked gently, aide to side and front to back, to mix the contents. The syringe plunger is drawn up to pull the cell suspension into the syringe and pushed back down again, mixing the cells. This repeated 1-2 times will provide an adequate mix in the syringe. After the 2nd or 3rd thaw the liquid level is brought to the 45 nil mark on the syringe The port is removed from the syringe, raised above the bag and closed. The suspension in the syringe is released into a 50 ml tube, which is closed. The culture bag port is reattached to the syringe and the process is repeated until the required amount is drawn off, The number of 50 ml tubes required will depend upon the volume to be drawn off. These cells will be frozen for further experiments, in vials or in bulk, for future culture bags.

A sample must be removed from the culture bag, at this point, to provide Day 0 samples of cells and supernatant, for later cytotoxicity and cytokine assays. Usually, 40×10⁶ cells are removed. At 2×10⁶ cells/ml, this is approximately 20 mls. This sample can be removed, in the same manner, as excess cells were removed, including rocking the bag gently to mix. A 30 ml syringe can be used in place of the 60 ml syringe. The cells removed arc placed in a 50 ml tube. They are spun at 400 g (1200 rpm) for 10 min. 6 mls of the supernatant is saved and aliquoted into 1 ml sterile labeled vials and frozen at −20° C. The remaining supernatant is discarded. The cells are resuspended in 4 ml Freezing media (Fetal Bovine Serum+10% Dimethysuloxide) to give a concentration of 10×10⁶ cells/ml. 1 ml is aliquoted into each of 3 sterile, labeled cyrovials. 1 ml of Freezing media is added to the remaining 1 ml cell suspension to provide a 5×10⁶ cell/ml concentration, 1 ml is aliquoted into each of 2 sterile, labeled cryovials. A styrofoam box containing a styrofoam rack is kept in the −85° C. freezer at all times. All cryovials will be placed into this container until the next day when they will then be placed into the −135° C. freezer. The location (freezer rack, box and position) of these vials are written on a 3×5 card with the trial#-patient#-aphoresis#. date, day of incubation, cell #, volume and cap color (if necessary). These cards are kept in the Clinical Trial Inventory Box.

The ‘mucin’ peptide and Interluekin-2 (IL-2) is now added to the bag: Since the amount added of both depends upon the volume of the bag, the number of mls drawn off or added is carefully recorded in the patient book. The ‘mucin’ concentration in the bag is 1 ug/ml. The stock solution of the ‘mucin’ is 1 ug/ul so the amount of uls added to the bag is equal to the number of mls in the bag. The 1-2 cytokine concentration in the bag is 100 IU/ml. Since the stock solution concentration of IL-2 can vary, the total number of IU required must be determined by multiplying the volume of the bag (in mls)×100 IU/ml. This total number of IU is then divided by the stock solution concentration to give the number of uls needed. Both of these can be added using the same arrangement with a syringe acting as a funnel. A 3.5 or 10 ml syringe can be used. The plunger is removed before attaching the bag port and the ‘mucin’ and IL-2 are added using separate, sterile, individually wrapped micro pipet tips as far down in the syringe as possible. 2 ml of fresh AIM-V media is used to rinse down the syringe and bag tubing. This 2 ml is added to the bag volume total.

The bag can be mixed gently, as before, by rocking side to side and front to back. It is then placed in the 37° C. incubator with 5% CO2.

On Day 3: The bag should be rocked gently to mix, before the sample is drawn from the bag. Using a 10 ml syringe, after drawing and pushing back several times bring the level in the syringe to the 1 ml mark. After the port has been raised and closed, this sample can be put in a nonsterile vial to be taken for a CBC and then discarded. If the concentration is above 2×10⁶ cells/ml, media should be added to bring the concentration down. 40×10⁶ cells should then be removed and frozen for later assays, as on Day 0. 6 mls of supematant will be aliquoted, at 1 ml/vial, and frozen at −20° C., as on Day 0. Volume, added or removed, should be recorded. IL-2 is added in the same manner us before, determining the total IU required and dividing by the stock solution concentration.

On Day 7: 1 ml is drawn from the bag, as on Day 3, for a CRC. The cell concentration is determined and any media should be added. Sterility samples (13 mls) are removed on this day. The ‘mucin’ peptide and IL-2 are added as they were on Day 0, including any addition/subtraction of bag volume.

STERILITY: 13 mls are divided into 2 parts, 8 mls and 5 mls. The 5 ml aliquot of cells and supernatant, is placed into the Micro Test, Inc. M4-RT tube (Multi Micro Media). It is labeled with the trial#-patient#-aphoresis# and date. This is placed into the zipper pocket of the biohazard bag provided and the yellow form is put in the back pocket. The bag is taken to HCC and the courier is called to pickup for overnight shipment to Specialty Labs for PCR mycoplasma testing. Results are noted in patient book.

The 8 ml aliquot is spun at 400g (1200 rpm) for 10 min. The supernatant is decanted into a sterile tube labeled with the trial#-patient#-aphoresis#. 2 mils are removed to a separate sterile tube (for HARDY Diagnostics TSB and Fluid Thioglycollate tubes). The remainder, in the tube, is placed with cold packs into a styrofoam box. The necessary forms are copied, put in a plastic zipper bag and sealed in with the sample. The box is shipped overnight, via Airbourne, to Biowhittaker for endotoxin levels by LAL testing. Results are noted in patient book.

Of the 2 mls of supernatant removed previously, 1 ml is inoculated anaerobically to one of each, labeled TSB and Fluid Thioglycollate tube. This is done by gently placing the sample filled 1 ml pipet at the bottom of each tube. The sample in the pipet is then pushed slowly out to settle at the bottom of the tube. The pipet is carefully withdrawn to prevent any air bubbles being introduced into the fluid and the tubes closed tightly. An uninoculated tube of each kind, is also labeled as the other tubes. The Fluid Thioglycollate tubes are then placed into a 34° C. incubator and the caps loosened. The TSB tubes are placed in a room temperature cabinet. The tubes arc checked after 3, 7 and 14 days of incubation. Signs of turbidity indicate bacterial growth. The results are noted in the patient book.

On Day 8: After mixing the bag, sufficient volume is drawn off to provide enough cells and supernatant for the cytotoxicity and cytokine assays as well as frozen samples (40×10⁶) as on Day 0.1 ml is also removed into a sterile tube to be taken to BSA for a Gram Stain, identified by a patient label. The cell count from the previous day (Day 7) can be used to determine total cell number and volume needed to be drawn off. Usually 1×10⁶ cells are needed for the XTT and Almar Blue cytotoxicity assay. The cells are centrifuges at 400 g (1200 rpm), usually) for 10 min. and at least 6 mls of the supernatant is aliquoted into 6 vials with 1 ml/vial. The cytoxicity assay will be started as early as possible. (See XTT and Alamar Blue procedures). The cytokine assays (usually g-IFN and IL 10) will be started as soon as possible. (See cytokine procedure). Results from the cytokine assays should be available within six hours to determine infusion criteria.

Criteria for Infusion: Statistically significant increase of the Day 8 sample over the Day 0 sample in either of the cytokine assays run will be sufficient evidence for CTL production and infusion may proceed as long as the Gram Stain results are negative. If there is no statically significant increase in either cytokine assay then the infusion will be delayed until the XTT results are compiled on the next day. Assay data is provided to Dr. Stephen Wright for consultation/approval. Nancy Blades is contacted ASAP to determine timing of the infusion. Harvesting and preparing the cells (see Harvest Procedure) requires 2-3 hours.

### CTL Harvesting Standard Operating Procedure

Once harvest has started, one person will remain in the lab at all times.
1) Record Lot# and Expiration Date of all solutions on Harvest Form. Unless otherwise specified, all procedures will be preformed in the laminar flow hood. All used supplies will be discarded into the biohazard waste.
2) Place a 5-ml syringe in the support clamp. Remove the culture bag from the incubator and rock bag gently, side to side and front to rear, to mix. Swab bag port/cap with alcohol swab. Remove cap, laying it on the alcohol swab, and connect port to syringe. Release tubing clamp and manipulate tubing to reduce pinch caused by the clamp.
   a) Draw cell suspension into syringe by pulling the plunger slowly to the full volume of the syringe. Depress plunger slowly to return suspension to bag. Repeat twice.
   b) Draw plunger up again, gently, and bring down to the 2-ml mark.
   c) Disconnect from syringe; raise port to allow liquid to flow back into bag and then recap the port. Tighten tubing clamp.
   d) Dispense 1-ml of sample in the syringe into a sterile vial to be taken to BSA Microbiology Laboratory for a Gram Stain. A result of “No organisms seen” from the Gram Stain must be received, via voice pager, before the cells can be given back to the patient This message will be heard and understood by both techs and recorded on the Harvest form.
   e) Dispense the remaining 1 ml of the sample into a micro-centrifuge tube to be taken to Harrington Cancer Center Laboratory for CBC.
   f) Remove the 5 ml syringe from the support clamp and dispose of it in waste.
3) The number of cells to be harvested is determined by the patient's body surface area: 1-4×10⁸ cells/m². The required number of cells arc removed front the bag by the following method:
   a) Determine the volume to be removed from the bag (the number of cells required divided by the number of cells per ml in the bag (from CBC)).
   b) Place a 60-ml syringe in the support clamp Mix bag gently, as before, and prop labeled up with a low rack or box.
   c) Swab bag cap/part with alcohol swab. Remove cap and attach port to syringe, as before. Release tubing clamp, manipulating tubing to lessen pinch. Tightening clamp on a different part of the tubing each time will allow tubing to ‘recover’.
   d) Draw cell suspension into syringe, as before (3×). Draw up last time and bring down to 45-ml mark
   e) Disconnect the bag port, raise and cap. Close tubing clamp.
   f) Dispense cell suspension into sterile 50-ml tube and cap tube snuggly:
   g) Repeat from “c)” as needed to draw out appropriate amount of cell suspension.
4) Return the culture bag to incubator.
5) Balance all tubes in centrifuge holders. Use a balance tube, if necessary. Spin 1200 rpm (400×g) for 11 min. Brake is on.
6) While cells are centrifuging:
   a.) Transfer 25-ml of 25% v/v albumin to an infusion bag, using a syringe/needle, making sure to swab all connections before and after with alcohol.
   b) Transfer about 250 ml of 0.9% NaCl into a sterile bottle or flask, using psyringe/needle or ‘tubing’ set, making sure to swab all connections before and after. Add 50 ml of NaCl to the infusion bag using a syringe/needle, making sure to swab all connections before and after with alcohol.
7) After centrifuge stops, remove tubes carefully from carriers and return to hood.
8) Aspirate the supernatant, using a vacuum/suction system. The vacuum flask will have 100 ml of ‘Bleach’ in it.
   a) Attach a sterile, glass Pasteur pipet to the suction flask tubing.
   b) Care should be taken since the pellet will be soft. Keeping the tube upright, aspirate the supernatant down to the cone in the tube, leaving several mls in bottom.
   c) After aspirating all tubes, suction bleach up into pipet, from a separate small flask, to clean it and the tubing.
9) Each of the tubes has a cell pellet and all of these pellets must be combined into one.
   a) Add 25-ml of AIM-V media to first tube and gently resuspend the pellet by releasing the media over the top of the pellet. After all of the media is dispensed, draw it up into the pipet and release again. Repeat until the pellet is completely broken apart.
   b) After the cell pellet is broken, draw up the 25 ml of cell suspension and add it to the next tube. Repeat the process of resuspension using the same 25 ml for all of the tubes.
   c) Using 10 ml fresh AIM-V media, rinse all tubes and add wash to previous 25 ml cell suspension.
10) Balance tube and centrifuge at 1200 rpm (400 g) for 11 min. Brake is on.
11) When centrifuge has stopped, aspirate supernatant.
12) Wash cells once in 0.9% NaCl by resuspending the cell pellet in 25 ml of the saline.
13) Balance tube and centrifuge at 1200 rpm (400 g) for 11 min. Brake is on.
14) Aspirate supernatant. Resuspend the cell pellet in 25 ml of 0.9% NaCl. Draw up this cell suspension in a 30-ml syringe/needle and inject into the infusion bag, making sure to swab all connections before and after with alcohol.
15) Add 25 ml more of 0.9% NaCI to the tube using it to rinse the sides and bottom. Once tube is rinsed, draw off the wash with a 30-ml syringe/needle and inject into the infusion bag, making sure to swab all connections before and after with alcohol.
16) Gently mix the contents in the infusion bag by tilting the bag.
17) Attach a label with the patient's name, number of cells, amount of fluid in bag, and protocol-patient and aphoresis number.
18) The infusion bag is then taken to Harrington Cancer Center and given to the nurse in charge of our patient. Both technician and nurse wilt sign the Tracking Form. A copy of will be given to Nancy Blades and the original will be placed in the patient's laboratory notebook.

All publications, patents, patent applications, and other documents mentioned in the specification are indicative of the level of those skilled in the art to which this invention pertains. All publications, patents, patent applications, and other documents are herein incorporated herein by reference in their entirety for all purposes to the same extent as if each individual publication, patent, patent application, or other document was specifically and individually indicated to be incorporated herein by reference in its entirety for all purposes. Subheadings are included solely for ease of review of the document and are not intended to be a limitation on the contents of the document in any way.

Although the foregoing invention has been described in some detail by way of illustration and example for purposes of clarity of understanding, it will be obvious that certain changes and modifications may be practiced within the scope of the appended claims.

What is claimed is:

1. A method for detecting and localizing a cell-specific antigen in a mammal comprising the steps of:
(a) obtaining peripheral blood mononuclear cells from said mammal,
(b) exposing said peripheral blood mononuclear cells to a peptide that displays an immunogenic epitope of said cell-specific antigen under conditions such that T lymphocytes in said peripheral blood mononuclear cells undergo antigen-specific activation, thereby producing antigen-specific T lymphocytes that bind to said cell-specific antigen;
(c) labeling said antigen-specific T lymphocytes with a label that is detectable by imaging, thereby producing labeled antigen-specific T lymphocytes,
(d) administering said labeled antigen-specific T lymphocytes to said mammal, and
(e) determining the distribution of said labeled antigen-specific T lymphocytes in said mammal by imaging, thereby detecting and localizing said cell-specific antigen in the mammal.

2. The method of claim 1 wherein the step (b) of exposing said peripheral blood mononuclear cells to said peptide is performed in the presence of interleukin-2 (IL-2).

3. The method of claim 1 wherein the step (b) of exposing said peripheral blood mononuclear cells to said peptide is performed by adding a cell-free preparation of said peptide to said peripheral blood mononuclear cells and without adding additional cells to said peripheral blood mononuclear cells prior to step (d) of administering said labeled antigen-specific T lymphocytes to said mammal.

4. The method of claim 1 wherein said antigen-specific T lymphocytes are cytolytic for cells that express said cell-specific antigen.

5. The method of claim 1 wherein said antigen-specific T lymphocytes comprise CD4+ lymphocytes.

6. The method of claim 1 wherein said antigen-specific T lymphocytes comprise CD8+ lymphocytes.

7. The method of claim 1 wherein said antigen-specific T lymphocytes comprise CD45RO+memory T cells.

8. The method of claim 1 wherein said antigen-specific T lymphocytes comprise negligible amounts of natural killer (NK) cells.

9. The method of claim 1 wherein step (d) of administering said labeled antigen-specific T lymphocytes to said mammal is performed without administering IL-2 to said mammal with said T lymphocytes or thereafter before performing step (e) of determining the distribution of said labeled antigen-specific T lymphocytes in said mammal.

10. The method of claim 1 wherein step (d) of administering said labeled antigen specific T lymphocytes to said mammal comprises administering said lymphocytes intraperitoneally.

11. The method of claim 1 wherein step (d) of administering said labeled antigen specific T lymphocytes to said mammal comprises administering said lymphocytes intravenously.

12. The method of claim 11 wherein administering said lymphocytes intravenously comprises administering a glycoconjugate to said mammal such that said trafficking of said lymphocytes is altered compared to administering said lymphocytes without administering said glycoconjugate to said mammal.

13. The method of claim 11 wherein administering a glycoconjugate comprising administering a glycoconjugate comprises administering asialoorosomucoid.

14. The method of claim 11 wherein administering a glycoconjugate comprising administering a glycoconjugate comprises administering orosomucoid.

15. The method of claim 1 wherein said method wherein said cell-specific antigen is a tumor-specific antigen.

16. The method of claim 10 wherein said peptide displays an epitope of human mucin1 (MUC-1)

17. The method of claim 11 wherein said antigen-specific T lymphocytes comprise CD4+ lymphocytes exhibit MHC unrestricted cytotoxicity for cells bearing said epitope of MUC-1.

18. The method of claim 12 wherein said peptide displays an epitope of MUC-1 comprises an amino acid sequence that is a circular permutation of a MUC-1 sequence comprising the sequence PDTRP.

19. The method of claim 13 wherein said peptide has the amino acid sequence GSTAPPAHGVTSAPDTRPAP.

20. The method of claim 1 wherein said label is selected from the group consisting of a gamma emitter, a positron emitter, a magnetic material, a density based contrast material, and mixtures thereof.

21. The method of claim 20 wherein the label is a gamma emitter selected from the group consisting of indium-111, technetium-99m, technetium-99, iodine-123, and mixtures thereof.

22. The method of claim 21 wherein the label is indium-111.

23. The method of claim 1 wherein said imaging is selected from the group consisting of radioimaging, magnetic resonance imaging, positron emission tomographic and X-ray computed tomographic imaging.

24. The method of claim 23 wherein said imaging is selected from the group consisting of a single scan and serial scans.

25. The method of claim 23 wherein said imaging comprises a total body scan of said mammal.

26. The method of claim 23 wherein said imaging comprises at least two separate scans wherein each of said separate scans is selected from the group consisting of positron emission tomographic and X-ray computed tomographic imaging.

27. The method of claim 26 wherein imaging data obtained from two or more separate scans are compared.

28. The method of claim 26 wherein imaging data obtained from two or more separate scans are fused into a single display image.


SEQUENCE LISTING (4 sequences)

SEQ ID NO: 1 - protein, 5 aa, Homo sapiens
PDTRP

SEQ ID NO: 2 - protein, 20 aa, Homo sapiens
GSTAPPAHGVTSAPDTRPAP

SEQ ID NO: 3 - protein, 24 aa, Artificial
  Believed to be based on MUC-1 sequence of Homo Sapiens; see U. S. Patent No. 6,600,012
STAPPAHGVTSAPDTRAPGSTAPP

SEQ ID NO: 4 - protein, 12 aa, Homo sapiens
PGSTAPPAHGVT